(19) United States
(12) Patent Application Publication
(10) Pub. No.: US 2026/0133978 A1
(43) Pub. Date: May 14, 2026

(54) GAIT-BASED BIOMETRIC DATA ANALYSIS SYSTEM

(71) Applicant: AUTONOMOUS ID CORPORATION, Ottawa (CA)

(72) Inventor: Todd GRAY, Ottawa (CA)

(21) Appl. No.: 19/438,900

(22) Filed: Jan. 2, 2026

Related U.S. Application Data
(63) Continuation-in-part of application No. 19/258,580, filed on Jul. 2, 2025.
Application No. 19/258,580 is a division of application No. 16/270,959, filed on Feb. 8, 2019, now Pat. No. 12,380,114.
Application No. 16/270,959 is a continuation of application No. 15/826,744, filed on Nov. 30, 2017, now Pat. No. 10,885,041.
Application No. 15/826,744 is a division of application No. 14/946,358, filed on Nov. 19, 2015, now Pat. No. 9,864,780.
Application No. 14/946,358 is a continuation-in-part of application No. 13/939,923, filed on Jul. 11, 2013, now Pat. No. 9,204,797.
Application No. 13/939,923 is a continuation-in-part of application No. 13/581,633, filed on Dec. 6, 2012, now Pat. No. 9,188,963.

(51) Int. Cl. G06F 16/2457; A43B 3/34; A61B 5/00; A61B 5/103; A61B 5/11; G06F 16/248

(52) U.S. Cl. CPC G06F 16/24575; A43B 3/34; A61B 5/1038; A61B 5/112; A61B 5/6807; G06F 16/248; G16H 10/40; G16H 10/60; G16H 50/20; A61B 2562/046

(57) ABSTRACT

The present invention discloses an AI-powered biometric identity and gait-based disease monitoring system/platform. The system captures gait data through wearable devices, analyzes gait metrics with vertical disease-specific AI models and horizontal pharmacological AI models. The system may also generate biometric identity hashes, and is able to continuously track disease progression through longitudinal AI analysis. The system further integrates drug-gait interaction modeling and secure cloud deployment while generating clinician-ready reporting formats. The system may also be used to continuously assess insurance-related matters such as premiums and policy-related actions.

### RELATED APPLICATIONS

This application is a Continuation-in-Part of U.S. patent application Ser. No. 19/258,580 filed Jul. 2, 2025, which is a Divisional of U.S. patent application Ser. No. 16/270,959 filed Feb. 8, 2019, which is a Continuation of U.S. patent application Ser. No. 15/826,744 filed Nov. 30, 2017, which is a Divisional of U.S. patent application Ser. No. 14/946,358 filed Nov. 19, 2015, which is a Continuation-in-Part of U.S. patent application Ser. No. 13/939,923 filed Jul. 11, 2013, which is a Continuation-in-Part of U.S. patent application Ser. No. 13/581,633 filed Dec. 6, 2012.

### TECHNICAL FIELD

The present invention relates generally to biometric identity systems and clinical monitoring technologies. More particularly, the invention pertains to an artificial intelligence (AI) based system that allows for biometric identification, personalized chronic disease monitoring, pharmacological interaction modeling, longitudinal gait tracking, and secure healthcare data management. The system provides an AI-centric analytical system based on biometric loop signature formation with data analysis being performed in conjunction with an insole insert in a shoe for personalized analytics or in a populated data system for movement and mobility research in general.

### BACKGROUND

The increase in activity in the personal wellness and fitness, mobile healthcare and user discrete analytical fields have highlighted some shortcomings of current personal and population based analytics systems as well as the effects of generally prescribed pharmacological and therapeutic treatments after diagnosis. One of the fields that has been given attention in the wellness and fitness field is gait analysis.

Gait analysis is commonly used for diagnostic and therapeutic monitoring of patients with neuromuscular, orthopedic, metabolic, and aging-related disorders. However, conventional systems typically rely on subjective assessments, limited disease targeting, and siloed data collection. Similarly, prior biometric identity systems fail to leverage multi-dimensional clinical insights from gait parameters. Current technologies lack AI-driven personalization, integration of pharmacological side-effect modeling, and continuous longitudinal monitoring tied to both biometric identity and clinical conditions.

There remains an unmet need for a system that unifies biometric identity, individualized chronic disease monitoring, and pharmacovigilance analysis through adaptive AI models trained on personalized gait metrics, medication histories, comorbidities, and demographic data. There is, therefore, a need for an analytical system that is neither invasive nor linear in scope and use and which provides access and analysis for data gathered by convenient user-worn devices.

### SUMMARY

The present invention discloses an AI-powered biometric identity and gait-based disease monitoring system/platform. The system captures gait data through wearable devices, analyzes gait metrics with vertical disease-specific AI models and horizontal pharmacological AI models. The system may also generate biometric identity hashes, and is able to continuously track disease progression through longitudinal AI analysis. The system further integrates drug-gait interaction modeling, secure cloud deployment, and generates clinician-ready reporting formats. The system may also be used to continuously assess insurance-related matters such as premiums and policy-related actions.

The invention allows for real-time, individualized AI decision-making tailored to each user's medical history, active medications, comorbid conditions, ethnicity, gender, and disease baseline metrics.

The present invention provides AI-based analytical systems and methods for the assessment of movement and mobility based on gait as well as the assessment of user health and welfare conditions based on the user's gait. A sensor component with multiple sensors is placed inside a user's shoe and biometric data is gathered from the sensors when the user takes a step or walks. The data is used to generate loops as the various sets of data are plotted against each other to form a loop based biometric of a user. The loops obtained from the data are then compared against stored loops previously obtained as well as other characteristic data extrapolated from the loops. Based on the results of the comparison, the user's movement and mobility characteristics are assessed using predetermined indicators in conjunction with analytical input from distributed databases and with the input of each user's specific characteristic data from which other data can be extrapolated. Using the biometric data, it can be determined whether the user has increased activity and performance or whether the user has the proper fitting insole insert and, if not, recommendations for production alteration can be made. The data analysis, as well as the comparison between the previously gathered data/loops, is performed using trained AI models. The trained AI models are also used such that the system can determine, based on the gathered gait data, whether the user has a specific condition or ailment, whether a specific condition or ailment is worsening, or whether a specific condition or ailment is improving.

In one embodiment, the user's biometric data is, preferably, previously extracted from his gait. The previously gathered data, and the plotted loops derived therefrom, can be used as a baseline for the user. Subsequent biometric data sets gathered from the user can then be compared against the baseline. Depending on the comparison results, a range of AI based analyses can be performed to determine metrics including progression or regression of a user's movement and mobility. Data gathered from the general population can be used to establish any correlation between a person's changing gait as he or she progresses or regresses in a specific health and fitness condition. Treatment effects of prescribed pharmacological and/or therapeutic remedies can also be determined using the baseline biometric data from the user along with AI trained models as the user progresses in his or her daily programs and treatment. Periodic gathering of the user's biometric data can be used to track and monitor the effects of the treatment on the user's gait to establish any causal link between the treatment regime and the user's gait. Such links and the specific effects of the fitness program or treatment regime can then be used to further heighten the effectiveness of shoe based biometric data gathering devices as diagnostic tools. In addition to the above, the diagnostic tools can be supplemented by a network of distributed databases that have pathomechanical movement and mobility data. Such data, in conjunction with data received from an insole and with a user's specific characteristics, can, for example, be used to narrow down a suitable diagnosis for the user's pathomechanical abnormality. Such a network of distributed databases storing a population of movement and mobility data and related characteristic data such as foot shape, foot type, standing weight and posture can for example be used to narrow down gait based movement and mobility biomarkers associated to a range of performance, human ailments and disease. The conclusions from the gathered gait based data and the AI-based analyses can additionally be used to automatically administer and adjust insurance based metrics, coverage, premiums and other insurance related parameters.

In a first aspect, the present invention provides a method for determining changes in a user's movement and mobility using a foot-based gait device, said device having a plurality of sensors for gathering gait-based data, the method comprising:
a) selecting two of said plurality of sensors;
b) gathering data from each sensor selected in step a);
c) correlating data gathered from said two sensors such that data points gathered at similar instances are matched with one another to result in data pairs;
d) determining at least one characteristic loop from said data pairs, each characteristic loop being a loop formed when said data point pairs are plotted;
e) retrieving signature characteristic data, said signature characteristic data being derived from data resulting from biometric data previously gathered from said user;
f) determining a signature characteristic loop from said signature characteristic data;
g) comparing characteristics of said at least one characteristic loop determined in step d) with characteristics of said signature characteristic loop determined in step f);
h) in the event a comparison of said characteristics compared in step g) produces results not within predetermined limits, determining that a change has occurred in said user's gait-based biometric data;
i) in the event a comparison of said characteristics compared in step g) produces results within predetermined limits, determining that a change has not occurred in said user's gait-based biometric data.

In a second aspect, the present invention provides a system for diagnosing a change in a user's gait-based biometric data, the system comprising:
a sensor component comprising at least one sensor for gathering gait-based biometric data from said user
a data storage block for storing data relating to a signature loop, said signature loop being a loop resulting from a plot of data pairs derived from data gathered from said sensor component when said user uses said system
a data processing block for receiving data from said sensor component, said data processing block being for determining characteristic loops from said data received from said sensor component and for comparing characteristics of said characteristic loops with characteristics of said signature loop wherein
a change in said user's gait-based biometric data is indicated when said characteristics of said characteristic loops are not within predetermined limits of said characteristics of said signature loops.

In a third aspect, the present invention provides for use of an insole for use with a user's shoe to determine if a change has occurred in a user's gait-based biometric data, said insole comprising a plurality of sensors for measuring and detecting force applied by said user on different parts of said insole.

In a fourth aspect, the present invention provides for a network of distributed databases storing populated characteristic data for individual, group and general analytical insights in human movement and mobility.

In a further aspect, the present invention provides a method for determining changes in a user's condition using a foot-based gait device, said device having a plurality of sensors for gathering gait-based data, the method comprising:
a) selecting at least two of said plurality of sensors;
b) gathering data from each sensor selected in step a);
c) retrieving signature characteristic data, said signature characteristic data being derived from data resulting from biometric data previously gathered from said user;
d) determining characteristic data from said data from said sensors;
e) comparing characteristics of said characteristic data determined in step d) with characteristics of said signature characteristic data retrieved in step c);
f) in the event a comparison of said characteristics compared in step e) produces results not within predetermined limits, determining that a change has occurred in said user's condition;
g) in the event a comparison of said characteristics compared in step e) produces results within predetermined limits, determining that a change has not occurred in said user's condition;
wherein said foot-based gait device is an insole for gathering data regarding an individual's gait.

Another aspect of the present invention provides a system for use in determining at least one change in a user's condition, the system comprising:
a sensor component comprising at least one sensor for gathering gait-based biometric data from said user;
a data storage block for storing signature data, said signature data being data resulting from an analysis of data gathered from said sensor component when said user uses said system;
a data processing block for receiving data from said sensor component, said data processing block being for determining characteristic data from said data received from said sensor component and for comparing characteristics of said characteristic data with characteristics of said signature data;
wherein
a change in said user's condition is indicated when said characteristics of said characteristic data are not within predetermined limits of said characteristics of said signature data; and
wherein said sensor component comprises an insole for use with said user's shoe.

Yet a further aspect of the present invention provides for a use of an insole for use with a user's shoe to determine one or more changes in a user's condition, said insole comprising a plurality of sensors for measuring and detecting gait-based force applied by said user on different parts of said insole.

In another aspect, the present invention provides a system for assessing a user's conditions, the system comprising:
a wearable device comprising a plurality of sensors producing data readings;
a server configured to:
receive said data readings from said wearable device;
combine and organize said data readings into specific groupings based on time periods;
combine and organize said specific groupings into specific actions of said user over specific time periods;
compare prior and current data readings for said user using trained AI models to determine any changes in said user's condition.

Additionally, the server may be configured to compare prior and current data readings for said user using trained diagnosis AI models to determine one or more conditions of said user.

As another aspect, server may be configured to create a unique hash value based on at least one data reading, said hash value being for use as a user identifier.

In another aspect, the server is further configured to, in conjunction with data relating to said user's treatment regimen, compare prior and current data readings for said user using trained treatment AI models to determine effects of said treatment regimen on one or more conditions of said user.

In a further aspect, the server is configured to pass differences between said prior and current data readings through different trained diagnosis AI models, each diagnosis AI model being for determining if said differences indicate a presence of one or more conditions in said user.

As well, server may be configured to pass differences between said prior and current data readings through different trained treatment AI models to determine gait-based side effects of said user's treatment regimen.

In yet a further aspect, the server is configured to generate reports for said user's conditions based on analyses of said data readings and transmit said reports to health professionals.

Yet a further aspect provides that the unique hash value is based on one or more data readings from an immediately previous session with said user, said unique hash value being compared with a unique hash value derived from one or more current data readings.

In another aspect, trained AI models to be used in assessing said user's conditions are predetermined and based on at least one of:
said user's previous diagnosis;
said user's previously prescribed medications;
said user's previously assessed condition; and
said user's previously prescribed treatment regimen.

A further aspect of the present invention provides that the data readings are used by said server to determine one or more of:
stride length;
cadence;
gait speed;
stride variability;
center of force;
a balance score;
load-bearing weight;
standing weight;
step count; and
weight biomarker shifts.

Further to the above, the present invention may provide that the server is configured to monitor outputs of one or more of said trained AI models for automated model drift detection, wherein, when statistically significant deviations from predefined limits are detected in said outputs, said server triggers an alert.

According to another aspect of the present invention, there is provided a method for assessing a user's condition, the method comprising:
a) receiving data from a wearable device, said data comprising current data readings for said user;
b) retrieving said user's stored data based on said data received from said wearable device, said stored data comprising prior data readings for said user;
c) processing said data to compare prior and current data readings for said user to determine any changes in said user's condition;
wherein step c) is executed using trained AI models.

As another aspect, the server may be configured to:
ingest data readings and data analyses outputs for said user; and
calculate dynamic risk scores based on said data readings and said data analyses outputs for use in continuous underwriting of health or life insurance coverage for said user.

### BRIEF DESCRIPTION OF THE DRAWINGS

The embodiments of the present invention will now be described by reference to the following figures, in which identical reference numerals in different figures indicate identical elements and in which:

FIG. 1 is a block diagram of the system according to one aspect of the invention;

FIG. 2A is an image illustrating the different forces applied by a human foot as it takes a step;

FIG. 2B is a diagram illustrating different zones on an insole according to one embodiment of another aspect of the invention;

FIG. 3 illustrates raw data waveforms and data waveforms after a low pass filter has been applied;

FIG. 4 illustrates loops plotted using raw data and filtered data;

FIG. 5 illustrates a number of characteristics for different sets of data from the same user as well as an average characteristic loop derived from the other loops;

FIG. 6 illustrates the different characteristics which may be derived from the characteristic loops;

FIG. 7 illustrates characteristic loops using highly correlated data;

FIG. 8A shows average characteristic loops for different users;

FIG. 8B is a block diagram illustrating the data connections between various components of the system according to another aspect of the invention;

FIG. 8C is another block diagram for an AI-centric system residing in the server or servers that is used in conjunction with the various components of the overall system;

FIG. 8D is a block diagram of a diagnosis block according to one aspect of the present invention;

FIG. 8E is a block diagram of a treatment block according to another aspect of the present invention;

FIG. 8F is a block diagram of an insurance block according yet another aspect of the present invention;

FIG. 9 is a flowchart illustrating the steps in a method according to another aspect of the invention;

FIG. 10 is a flowchart detailing the steps in an AI-centric method executed by the system according to another aspect of the present invention;

FIG. 11 illustrates a composition aware decomposition pipeline that maps product identifiers to active ingredient(s), salt/polymorph, release mechanism, excipients, brand/manufacturer, site, and lot/batch metadata;

FIG. 12 is a block diagram of a hierarchical model that pools evidence across components, brands, sites, and lots to produce component level attributions and lot level signal detection;

FIG. 13 is a block diagram for a payer/actuarial dashboard for mobility aware risk modeling and formulary optimization based on component level attributions;

FIG. 14 is a block diagram for an R&D reformulation simulator that varies component selections and release profiles to forecast gait and weight outcomes with uncertainty bounds;

FIG. 15 is a block diagram of an identity decoupled end to end pipeline from ingestion through identity gating, immutable data products, ML staging, and audit;

FIG. 16 is a block diagram of an immutable transform directed acyclic graph where each schema defined transform yields a content addressable object with cryptographic digest and parent pointer;

FIG. 17 is a block diagram of a sequence showing edge computed identity and scoped token issuance for downstream operations that does not involve embedding identity in data products;

FIG. 18 is a block diagram of a deterministic machine learning (ML) lifecycle with model registry, golden datasets, drift validation, and reproducible promotion;

FIG. 19 is a block diagram of an identity safe method for many to many evaluations across token scopes that produce matches and metrics without persistent identifiers;

FIG. 20 is a block diagram of a multi-device ingestion architecture showing various wearables (e.g., insoles, hearables, seeables, watches, torso patches, and medical grade devices (including defibrillator/ICD)) that stream normalized telemetry into a device abstraction layer for fusion with gait analytics; and

FIG. 21 is a block diagram of a device abstraction and capability registry system that maps device classes to supported modalities (e.g., pressure, IMU, ECG/PPG, respiration) and to transforms (feature extraction, exposure alignment, orchestration).

### DETAILED DESCRIPTION

The present invention, in one aspect, provides systems and methods relating to an intelligent AI-centric analytical system which uses user discrete characteristic data acquired from wearable and other mobile devices. The system has biometric authenticated data integrity, allows for personalized, group or population based analysis of characteristic data and is not vulnerable to legacy computing systems, power failures, or unauthorized system access. The system, especially the server connected to multiple health or fitness based databases, has the facility and learning capacity to assess and analyze a user's unique biometric loop signature data and other characteristics extrapolated from such loop signature data such as step count, step and stride length, stride to stride variability, and center of force at any time interval for comparison against stored user data. The analytical system has the capability for targeted analysis of groupings of populated data characteristics such as gender, nationality, age, height, weight, foot shape such as Germanic or Celtic, foot type such as high arch or flat arch, posture, health and fitness. This analysis may be performed for research in movement and mobility biomarkers as well as for research in relationships between treatment regimes and gait related effects.

For greater clarity, the various aspects of the present invention may incorporate or be interoperable with various types of wearable devices. While insole based sensing is a preferred embodiment, other wearable devices may interoperate with the insole-based device as well as other wearable devices. For even greater clarity, the term “wearable device” herein is device agnostic and encompasses, without limitation any and all of the following: foot based insoles and shoe embedded sensors; head worn devices (hearables such as earbuds/eartips; seeables such as smart glasses/visors); wrist worn watches/bands; chest/torso patches; belt or garment sensors; and medical grade or remote devices that stream physiologic telemetry, including external or implantable defibrillators (ICDs), pacemakers, ECG/PPG/SpO₂/BP/respiration sensors, and similar monitors. The various embodiments of the present invention fuses such signals with gait telemetry for the analytics disclosed herein.

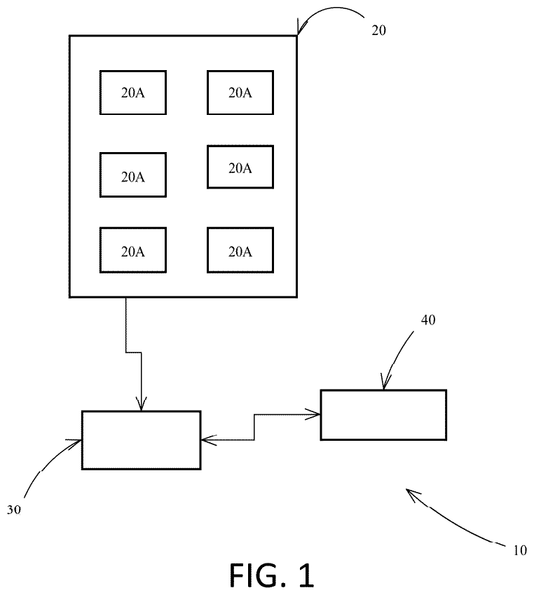

Referring to FIG. 1, a block diagram of one embodiment of the present invention is illustrated. As can be seen, the system 10 includes a sensor component 20 coupled to a data processing block 30 and which may receive data from a storage block 40. In broad terms, the sensor component 20, having multiple sensors 20A, generates biometric data from the sensors (biometric data based on the user's gait) which is then sent to the data processing block 30. The data processing block 30 then processes the biometric data and retrieves signature data from the storage block 40. The signature data comprises data that was previously gathered from the user who is currently being assessed. The data processing block then compares the signature data with the biometric data gathered from the multiple sensors. If there are differences between the newly gathered data and the previously gathered data, the data processing block then determines if the differences are in line with known patterns which would indicate notable events or assessments such as progression or regression of known user conditions. Other notable events or assessments can be determined by further analyses using remote data processing systems with such events or assessments including side effects from treatment regimes and the detection and/or diagnosis of new diseases or health conditions for the user. This conclusion would be based on gait-based biometric data which would indicate an improvement or decline in health and fitness or new health conditions.

In one implementation, the system includes a device abstraction layer that normalizes inputs from heterogeneous devices into a common schema (e.g., timestamps, sensor modality, sampling rate, calibration, quality flags, etc.) for downstream transforms. As well, the system may be configured to ingest telemetry from head, wrist, torso, and garment worn devices and from medical grade devices (e.g., defibrillator/ICD event logs, ECG/PPG waveforms, respiration/SpO₂), via secure local or remote links. In addition, where supported, identity primitives (e.g., device attestation, gait hash) may be computed per device at the edge (i.e., at the edge of the network) and federated through identity gating tokens as described herein. Furthermore, while the insole implementation remains a preferred embodiment for high fidelity foot kinetics, alternative embodiments may substitute or augment the insole with other wearables without departing from the scope of the invention.

As well, the system includes a communications block 50 that is coupled to the data processing block 30. The communications block 50 sends and/or receives communications regarding the comparison between the signature data and the data gathered from the sensors. The communications block 50 sends the data gathered to the data processing block 30 such that further data processing is performed remote from the user and/or the sensor component 20. The further data processing may be performed by a personal mobile device or by a server remote from the user's location and coupled to a network of distributed databases. By off-loading most of the post biometric authentication processing to a personal mobile device or to the remote server, the insole system does not need much processing capability.

It should be noted that the sensor component 20 has multiple sensors which gather data regarding a person's gait as well as other discrete user characteristics such as weight, stride length, and stride to stride variability. In one embodiment, the sensor component is an insole positioned inside the user's shoe, with the insole having multiple discrete force sensors that detect the amount of force or pressure exerted on a section or region of the insole. With multiple regions on the insole and at least one sensor positioned on each region, a user's gait can be profiled as being the amount of pressure that that user exerts on each region over time as the user takes a step. A variant of this sensor component would have at least one strain gauge positioned such that the pressure exerted on each of the multiple regions of the foot are detected by the gauge with each region corresponding to a section of the strain gauge. With such an arrangement, each section of the strain gauge thus acts as a different discrete sensor. In some variants, the sensor component also includes an accelerometer.

It should be noted that, in one embodiment, two insoles are used per user. This way, gait data may be gathered for each user foot. Data gathered from the user's left foot may be processed differently from data gathered from the user's right foot. The data gathered from each foot may then be combined to determine characteristics such as, for example, step count, cadence, velocity, and stride length. Alternatively, another embodiment only uses a single insole such that only one set of data is gathered per user. While the description below relates to a single insole, for a two insole embodiment, both insoles would be similar to one another and would, preferably, each conform to the description and principles outlined below.

For greater clarity, while two insole and single insole embodiments are detailed, the sensor component may alternatively be implemented by other wearable or medical devices as detailed above. In such cases, gait and mobility features are derived from available modalities (e.g., IMU from a watch or glasses; plantar pressure from an insole; ECG derived cadence from a chest patch) using the same feature extraction and longitudinal analysis described herein.

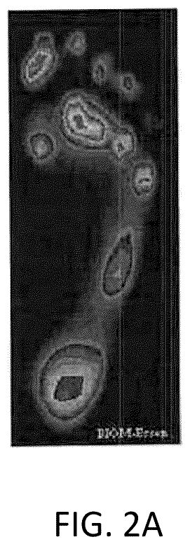

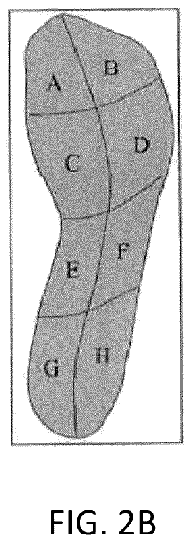

Referring to FIGS. 2A and 2B, a schematic illustration of a number of discrete pressure zones on an insole is illustrated. FIG. 2A shows an imprint of a human foot and the unique pressure points for a specific person. FIG. 2B illustrates the location of 8 specific pressure zones or areas on one embodiment of a pressure sensing insole. Each zone in FIG. 2B has a pressure sensing pad or sensor assigned to it such that the pressure exerted on each zone can be measured. A variant of this sensor component would have, instead of discrete sensor pads at each zone, a single strain gauge positioned as described above.

In the above embodiment, each sensor in the sensor component produces a signal linearly proportional to the force being applied to the sensor. Preferably, each sensor or zone would have a data channel dedicated to its readings for transmitting those readings to the data processing block. Alternatively, in one implementation, the readings can be time division multiplexed on to a single data line from the sensor component to the data processing block. In this implementation, the data is passed through a single A/D converter to produce multiplexed channels, one for each sensor. Of course, while there are eight zones in FIG. 2B, other variants may have more than eight zones or less than eight zones.

Regarding the data stream produced when the user is walking, in one embodiment, each sensor produces several hundred samples equating to approximately ten steps taken by the user. This data stream is then saved and examined by the data processing block and the actual step points are determined. Each step is identified, and the saved data stream resampled at a precise rate of approximately 100 samples per step.

It should be noted that multiple parameters regarding the user's gait can be extracted from the data produced by the sensor component depending on the type of sensors used in the sensor component. These parameters can then be used as points of comparison with the signature (or characteristic) data mentioned above. Some of these parameters may be:
Actual forces
Relative (normalized) forces.
Ratios between the peak forces in the eight sensor zones
Relative timing between forces on each sensor (strike and release sequence)
Average rate of change of force on each sensor zone
Maximum rate of change of force on each sensor zone
Frequency spectrum of the waveform from each sensor (ratio of values of harmonics derived from a Fourier transform)
Heel strike and toe lift off impact forces in the three axes.
Data waveform shape matching (waveform shape matching)

The parameters extracted from the data stream may then be compared directly or indirectly with the signature data noted above.

In one comparison scheme, the parameters extracted are used to derive a shape or loop, the characteristics of which can the compared with characteristics of a signature loop or shape. The use of a loop or shape allows for an indirect comparison between the data read by the sensor component and the signature or characteristic data. As well, it allows for more complex comparison schemes and for easier use of tolerances in the comparison.

For this comparison scheme, data from two different sensors are read by the data processing block. The two data sets (one from a first sensor and a second from a second sensor) are correlated with one another to synchronize the readings. This is done so that the data readings are synchronized in their time indices. Once synchronized, readings taken at approximately the same time index are matched with one another. Thus, the result is that a data reading from sensor A taken at time t1 is mated with a data reading from sensor B taken at time t1. The mating step results in a set of pairs of data readings from two different sensors.

It should be noted that a preferable preliminary step to the correlation step is that of applying a low pass filter to both sets of data. Such a low pass filter would remove the low frequency components of the signals and would provide cleaner and easier to process signals.

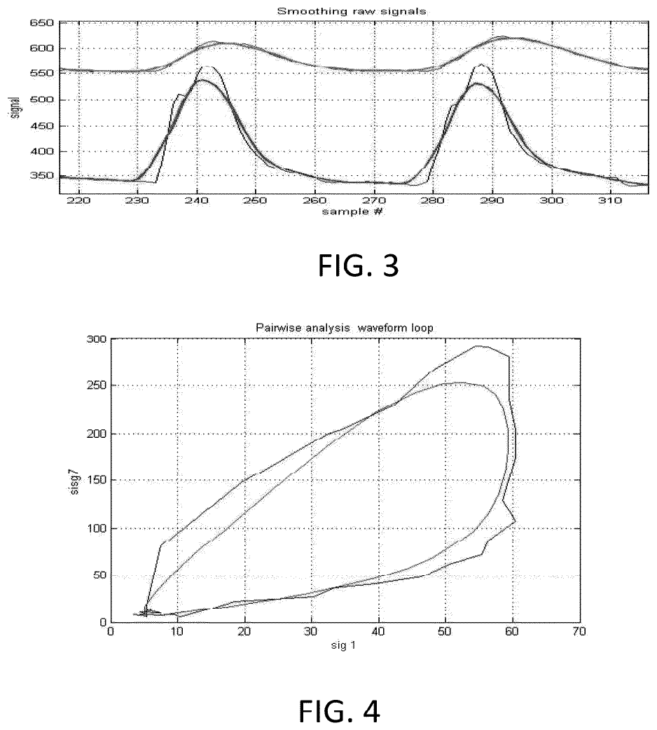

As an example of the processing performed on the data streams received from the sensor components, FIGS. 3-8 are provided to aid in the understanding of the process. Prior to any processing, data streams are first received from all of the sensors for a given fixed duration. For each sensor, the data stream for the given duration is saved by the data processing block. The resulting waveform for each sensor is then partitioned to determine discrete steps taken by the user. If the sensors are force/pressure sensors, this partitioning may be done by searching for peaks and valleys in the waveform. Each peak would denote a maximum force applied to the sensor and each valley would denote a minimum (if not absence) of force. Each step can then be seen as two valleys with a peak in between, representing the user's foot in the air, the actual step, and then user lifting his/her foot again. Alternatively, depending on how the system is configured, each step might be seen as two peaks bookending a valley.

Referring to FIG. 3, two raw data streams is shown at the bottom of the plot. After a low pass filter is applied to the signals, the smoother waveforms are shown at the top half of FIG. 3. From FIG. 3, one can see maximum force applied to the force pads for the two steps captured by the waveforms.

Once the discrete steps have been delineated in the data received from each of the sensors, each step for each sensor is then resampled to arrive at a predetermined number of data samples for each step. For the resampling, each sample is for a predetermined time frame and at a predetermined point in time in the current step. As an example, if each step lasts approximately 0.1 sec and 100 samples per step are desired, then the first sample is taken at the first one thousandth of a second in the waveform and the second sample is taken at the second one thousandth of a second and so on and so forth. This method essentially synchronizes all the samples such that it would be simple to determine all samples (from all the sensor readings) taken at the first one thousandth of a second or all samples taken at the first fiftieth one thousandths of a second as the relevant samples would all be similarly time indexed.

Once the different data waveforms from the different sensors have been synchronized, any two of the sensors and the data they produced can be selected for comparison with the signature data noted above and which may be stored in the data storage block. Depending on the configuration of the system, the signature or characteristic data stored in the data storage block may take numerous forms. In one example, multiple data sets/pairs (either filtered or as raw data) from the user may be stored so that a signature loop may be derived from the signature data whenever the characteristics of that signature loop are required. For this example, all the data pairs from all sensors would be stored so that any two sensors may be selected. Alternatively, the specific characteristics of the signature loop may be stored as the signature or characteristic data if one wanted to dispense with determining the signature loop every time a comparison needs to be made. As another alternative, only the data relating to the average signature loop derived from the user may be stored as signature data. Of course, if multiple sensors are to be used, then most possible average signature loops from the user data would be stored. In one other alternative, all the raw data (either filtered or not) from the user's steps may be stored as signature data. Such a configuration would allow for the greatest amount of flexibility as the system could randomly select any two of the sensors to be used and the signature data from the user would be available for those two sensors. As noted above, this configuration would require that the signature loop be calculated every time a comparison is required. The signature data may, if desired, be stored in encrypted format.

Once two of the sensors are selected from the sensors available in the sensor component (in this example the sensor component has 8 sensors, one for each of the eight zones illustrated in FIG. 2B), the resampled data for those two sensors are then mated with one another. This means that each time indexed sampling will have two points of data, one for the first sensor and another for the other sensor. These pairs of sensor readings can thus be used to create a characteristic loop. As an example, if sensors A and B are used and n denotes an index, then A[n] denotes the nth sampled reading from the waveform received from sensor A for a specific step. Similarly, B[n] is the nth sampled reading from the waveform received from sensor B for the same specific step. {A[n], B[n]} thus constitutes a data pair for the nth reading for that particular step. Plotting all the data pairs for a particular step, with readings from one sensor being on one axis and readings from the other sensor on the axis, results in an angled loop-like plot (see FIGS. 4-8 as examples). For pressure/force readings, this is not surprising as the force exerted by the foot in a particular step increases to a maximum and then decreases to as minimum as the person increases the weight the place on the foot and then removes that weight as the step progresses.

Once the data pairs have been created, a plot of the resulting loop can be made. As noted above, FIG. 3 shows the waveforms for two signals—the lower waveform being the raw data stream waveforms for 2 signals and the upper waveforms for the same 2 signals after a low pass filter has been applied. FIG. 4 shows a plot of the two sets of waveforms in FIG. 3. One loop in FIG. 4 is derived from the raw signal waveforms in FIG. 3 while the other loop is derived from the low pass filtered waveform in FIG. 3. As can be seen in FIG. 4, a smoother loop is produced by the low-pass filtered signals. It should be noted that the x-axis in FIG. 4 contains the values gathered from the first sensor selected while the y-axis contains the values gathered from the second selected sensor. It should be noted that while the embodiment discussed uses only a pair of sensors, the concept is applicable for 3, 4, or any number of sensors. If data from 3 sensors were used, then, instead of a 2D loop, a 3D loop may be created as a characteristic loop.

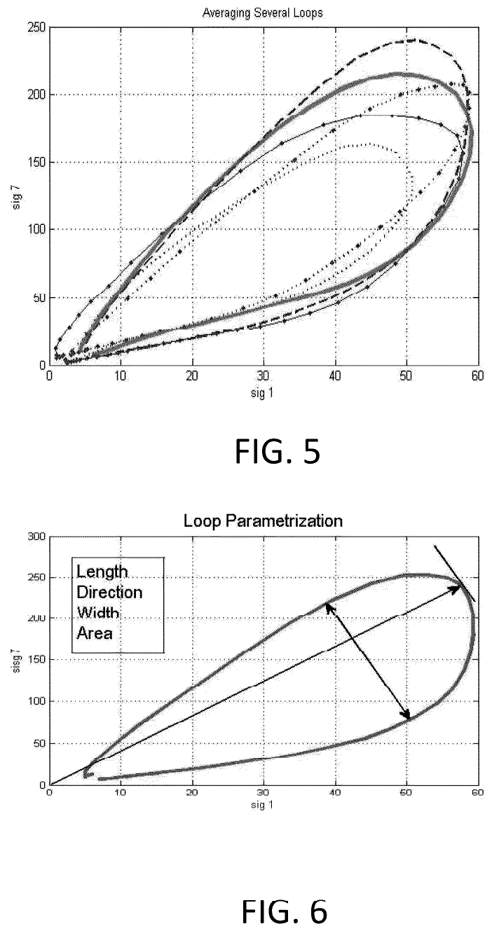

It should be noted that a loop can be formed for each one of the steps captured by the sensors. An averaged loop can be derived from the various loops formed from all the steps captured by the sensors. Referring to FIG. 5, the various loops from the various steps can be seen on the plot. An average loop (see darker loop in FIG. 5) is derived from all the loops captured using the low pass filtered waveforms. Multiple methods may be used to determine the average loop. However, in one embodiment, the points for the average loop are derived by averaging the various readings for each particular time index. As such, if the data pairs are as (An[i], Bn[i]) with An[i] denoting the nth reading for sensor A at time index i and Bn[i] denoting the nth reading for sensor B at time index i, then to derive the data reading for sensor A for the average loop for time index i, one merely averages all the An[i] where n=1, 2, 3, etc., etc. Similarly, for data reading for sensor B for the average loop for time index i, one merely averages all the Bn[i] where n=1, 2, 3, etc., etc. By doing this for all the multiple time indices, an average loop is derived from all the characteristic loops.

Once the average loop has been derived, the characteristics of that average loop can be determined. Referring to FIG. 6, some of the characteristics of the average loop can be seen. The length of the loop (measured from the origin), the width of the widest part of the loop, and the area occupied by the loop are just some of the characteristics which may be determined from the loop. As well, the direction of the loop (whether it develops in a clockwise or anti-clockwise manner) may also be seen as a characteristic of the loop. Another possible characteristic of the loop may be the angle between a ray from the origin to the farthest point of the loop and one of the axes of the plot. Additional characteristics of these loops may, of course, be used depending on the configuration of the system.

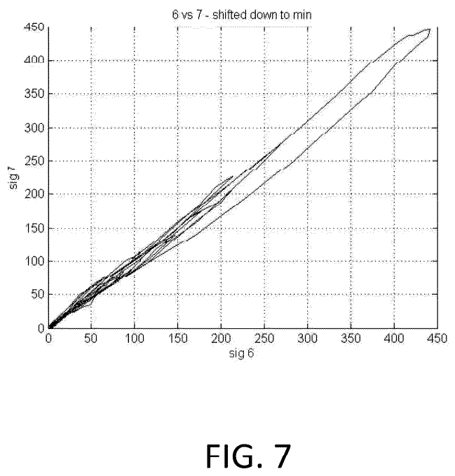

As another example of possible loops, FIG. 7 shows loops resulting from highly correlated data from the sensors. Such highly correlated data may produce loops that, at first glance, may not be overly useful. However, even such lopsided loops may yield useful characteristics. As an example, the amplitude from the furthest point may be used for an initial assessment of static of dynamic weight distribution.

Once the average loop for the steps captured by the sensors is determined, the characteristics for this average loop can be derived. Once derived, the same process is applied to the signature data stored in the storage block. The characteristics for the resulting signature loop (from the signature data) are then compared to the characteristics of the average loop from the data acquired from the sensors.

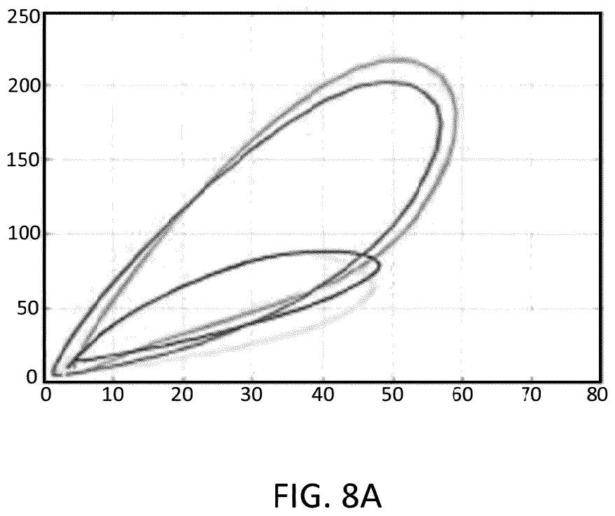

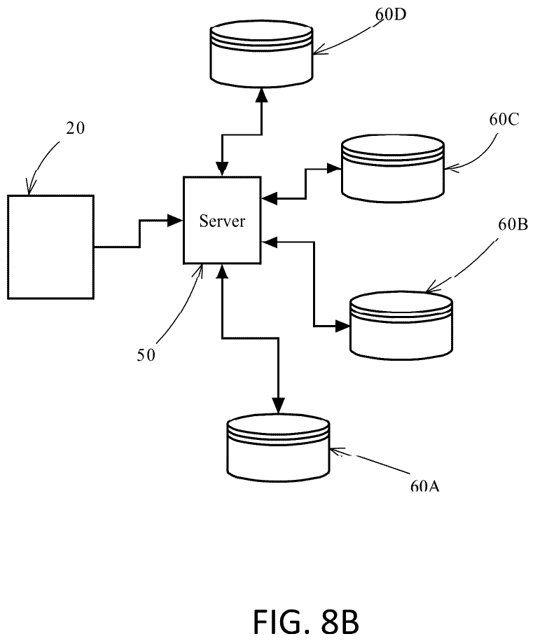

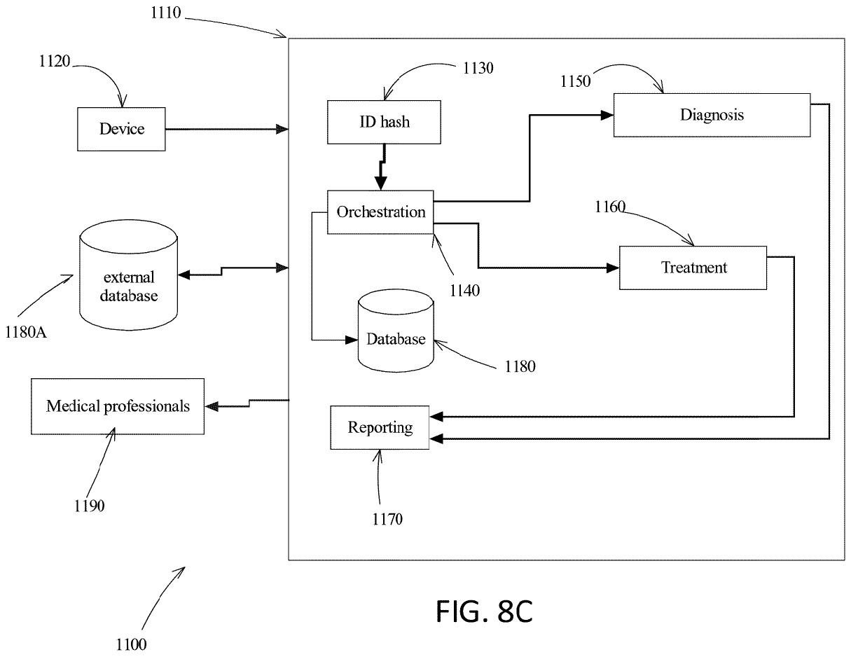

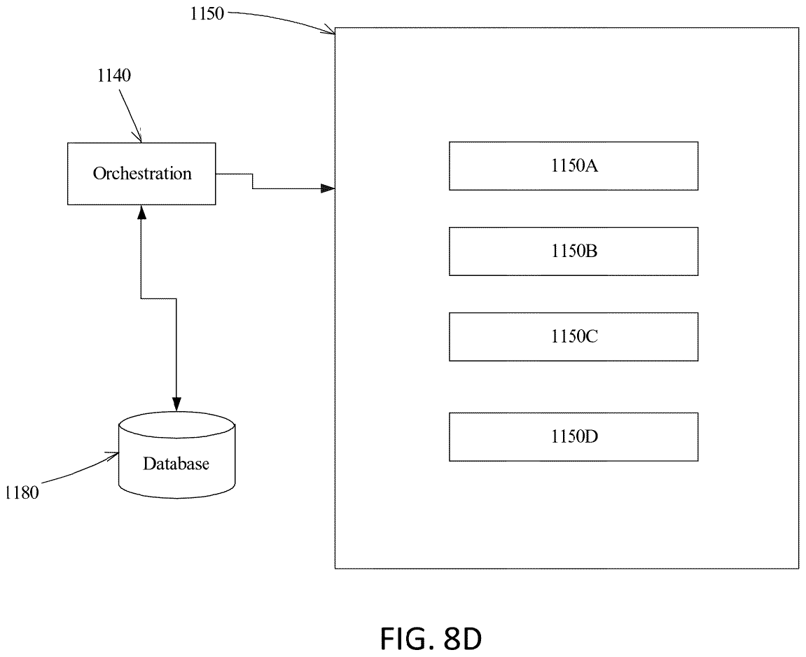

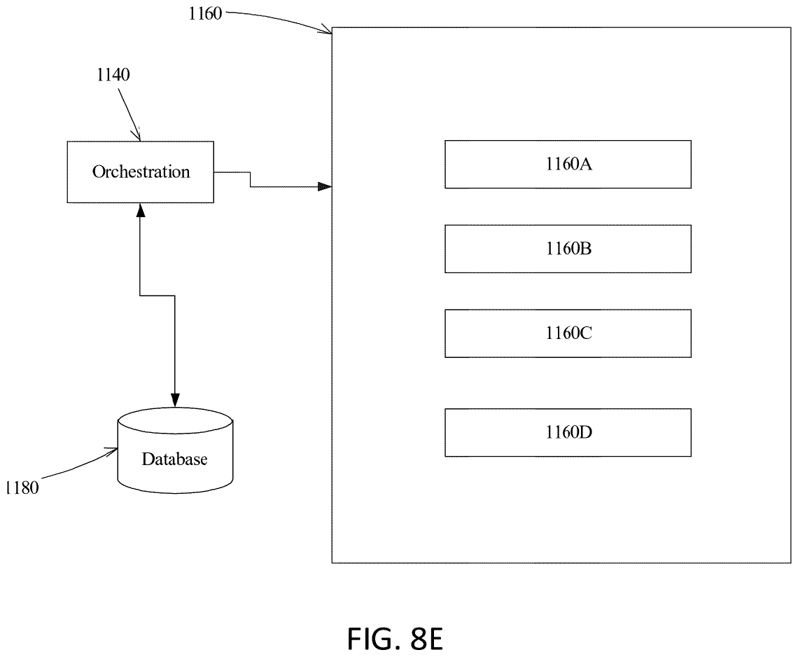

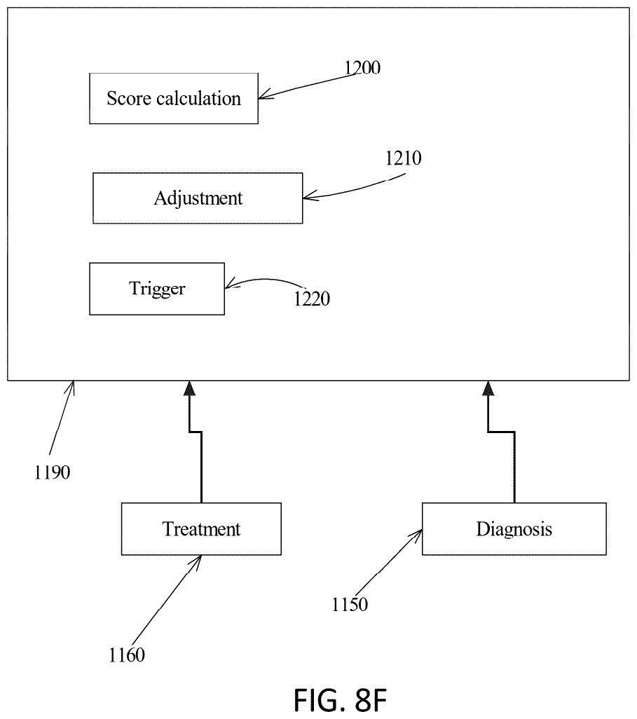

Referring to FIG. 8A, a comparison of two average loops from the gait of two individuals is illustrated. As can be seen, the characteristics of the two loops are quite different. One loop is clearly larger (more area), longer (length of loop), and wider (width at widest of the loops) than the other loop. It should be noted that custom tolerances can be applied to the comparison. Depending on the tolerance applied, the comparison can be successful (the characteristics match within the tolerances) or unsuccessful (even within the tolerances, there is no match). It should be noted that, as noted above, comparisons can be made for loops from a single user with data taken at different times. As an example, a user may have gait data taken at a first use of the system. Later gait data sets can then be taken for the same user at subsequent uses of the system. The loops derived from the initial gait data set and the subsequent gait data sets can then be compared to determine how a particular fitness program, treatment regimen, or physical condition has positively or negatively affected that user's gait over time.

Regarding tolerances, these can be preprogrammed into the system and can be determined when the signature data is gathered. As an example, a tolerance of 15% may be acceptable for some users while a tolerance of only 5% may be acceptable. This means that if the calculated characteristic of the average loop is within 15% of the calculated characteristic of the signature loop, then a match is declared. A match would indicate that there is no relevant difference between the loops being compared. Similarly, if a tolerance of only 5% is used, then if the calculated characteristic of the average loop is within 5% of the calculated characteristic of the signature loop, then a match is declared. Of course, if the calculated characteristic of the average loop is not within the preprogrammed tolerance of the calculated characteristic of the signature loop, then a non-match is declared. A non-match would indicate that there is a relevant difference between the loops being compared. A match may indicate that, for example, a fitness program, treatment regimen, or condition has not affected a user's gait between the time the first set of gait data was gathered to the time the second set of gait data was gathered. A non-match may, of course, indicate that the fitness program, treatment regimen, or condition has affected the user's gait.

It should also be noted that, in addition to the tolerances noted above, the system may use a graduated system of matches or matching. This would mean that a level of confidence may be assigned to each match, a high level of confidence being an indication that there is a higher likelihood that there is a match between the two sets of data derived from the average loop and the signature loop. A match can then be declared once the level of confidence assigned is higher than a predetermined level. A non-match can similarly be declared once the level of confidence is lower than a predetermined level. A level of indecision can be declared when the level of confidence is between the two preset levels for match and non-match. If a set of data falls within the gray area or an area of indecision between the two preset levels, then more data can be retrieved from the sensors and this data can be processed as above to arrive at a determination of a match or a non-match.

It should further be noted that, as an alternative, instead of matching or not matching two loops derived from a user's gait data, the amount of difference between the two loops can be determined. A significant difference between the characteristics of the two loops, preferably derived from data gathered from the same user using the same sensors in the sensor component at different times, would indicate a change of some sort. A significant difference between such two loops would indicate a significant change from the time the first data set was gathered to the time the second data set was gathered. As noted above, this could indicate that a fitness program, treatment regimen, or condition was having an effect on the user's gait. It may also indicate that a user's physical or medical condition is either progressing or regressing. The characteristics for which a difference may be found may, as noted above, include the size of the loops, the angle of the loops to one of the axes of the plot, the perimeter of the loops, the area covered by the loops, as well as other characteristics. A tolerance may, of course, be built into the comparison subroutine. As an example, if the tolerance is set at 2%, if a characteristic of two loops are within 1% (i.e. less than 2%) of each other (e.g. the sizes of the two loops) then no difference is concluded.

For greater clarity, the difference between two loops may be quantified and, depending on how great the differences are, alarms or other steps may be taken. As an example, if the area of a loop derived from a user's initial data set is compared with the area of a loop derived from a data set gathered a few months later, the differences may be significant. If there is no appreciable difference, then one can conclude that no change has occurred in the user's condition. If, on the other hand, the second data set has a much larger area (e.g. 25% greater area than the area covered by the loop from the first data set), this may indicate that the user is walking slower or that the user is placing more pressure on his feet with each step. Depending on the user's physical condition, this may indicate a progression (getting worse) or a regression (getting better) of that condition. It may also indicate that a fitness program or treatment regimen being used may not be effective. A threshold may thus be programmed so that if the difference in value of a characteristic being compared between two loops exceeds a specific amount or percentage, an alarm may be activated.

Regarding the programming or storage of the signature or characteristic data into the system, this is preferably done when the user first registers and wears the insole component of the system. This first data set can provide a baseline set of data to be used in comparison with subsequent data sets. This is done by having the user use the insole/sensor component by taking a specific number of normal steps. These steps are then captured in the system and are stored as signature/characteristic or baseline data. Once stored, the signature data can be retrieved and various characteristics of the signature data (by way of the signature loop) can be determined as described above. As described above, the signature data stored may take any number of forms. The signature data may be the raw data gathered from the user when s/he took the specific number of normal steps. Alternatively, the signature data may be the filtered version of the raw data or it may be the various characteristics of the various possible signature loops. Also, instead of the raw data which forms the waveforms, the waveforms themselves may be stored as signature data. The signature data may take any form as long as the characteristics of the signature loops may be derived from or be extracted from the signature/characteristic/baseline data.

As noted above, the system may include a server and a network of distributed databases connected to that server. The data gathered by the insole may be transmitted to either the server or to the databases. Such a server or its connected network of distributed databases may, to assist in the diagnosis of the user's condition for example, be used to consult with at least one medical or fitness database. Referring to FIG. 8B, a block diagram illustrating the connections between the various parts of the system is illustrated. As can be seen from FIG. 8B, the user's insole data gathering sub-system 20 communicates with a server 50. The server 50 serves as the main data processing and analytical unit to determine for example the user's physical or medical condition based, at least in part, on the data gathered from the user's gait. After the server receives gait data or the characteristic data from the insole 20 (perhaps from two insoles), this can be used by the server to determine the user's physical or medical condition. This includes determining the condition's progression, regression, or development. This may be performed by referring to one or more medical or fitness databases 60A, 60B, 60C, 60D. The server may receive both structured and unstructured data from the databases and/or from the user's insole. Preferably, the databases contain data relating to disease pathology, human performance, workforce industrial safety, human kinetics, sports performance, post-operative rehabilitation, therapeutics, and/or biometric data relating to injury and disease pathologies including conditions such as diabetes, Parkinson's disease, dementia, aging, and other vestibular disorders.

To assist the server in determining a diagnosis and/or a determination of the progression, regression, or change in the user's condition, the following characteristics of the user may be entered into the server and may be taken into account in any analysis: a foot type, general health, fitness level, type of gait, height, weight, age, gender, and/or nationality. Data entry into the server may be effected using various well-known means. As an example, such data may be transferred from a user's profile to the server. The server may then take such user data, in conjunction with the gait-based and gait-derived data, and analyze such data with data from the various databases. Then, based on the input from the various databases and the gathered data for the user, the server can produce its output.

The server's output may include an indication that the user's condition has regressed, progressed, is abnormal, or is normal. Similarly, the output may indicate the pathologies operative with the user as well as an indication if the user requires or is using corrective orthotics.

It should be noted that the user's foot characteristic may be one of: Egyptian, Roman, Greek, Germanic, or Celtic. Similarly, the user's foot type may be one of: flat arch, medium arch, or high arch. The user's type of gait may be one of: normal, toe in or toe out, knee in or knee out, lean forward or lean backward, posture easy or posture rigid, and trunk sway. The user's health and/or fitness may be categorized as one of: athletic, fit, average, below average, poor, or one where the user has a reported illness or disease.

The present invention may be configured such that, after the differences between a baseline loop and a current loop are determined, these differences are then assessed by one or more trained AI models. The assessment may be to determine the effects on the user's condition of any treatment regime the user is under as well as to determine the user's overall condition relative to the time the baseline loop data was gathered. The AI based assessment may also be used to determine the progression or regression of any gait related or gait affecting condition that a user may have.

The system of the present invention may comprise multiple software and hardware components operating within a unified architecture to accomplish biometric identity verification, disease monitoring, pharmacological analysis, and longitudinal progression tracking based on gait data. As noted above, the system utilizes wearable devices embedded with multi-dimensional gait sensors. The sensors capture and record various gait parameters including, but not limited to:
Stride length;
Cadence;
Gait speed;
Stride variability;
Center of force;
Balance score;
Load-bearing weight;
Standing weight;
Step count; and
Weight biomarker deviation.

In addition to the above, in some embodiments, the system of the present invention may fuse/integrate physiologic streams from non insole devices—including ECG, PPG, SpO₂, blood pressure (cuff or cuffless), respiration, skin temperature, EDA, EMG, and defibrillator/ICD event telemetry—to refine mobility state estimation, drug effect attribution, and alerting.

The system may be configured to include a biometric identity hash engine. The engine is used to generates a unique biometric identity hash from the gait data. This is done by processing gait data into an irreversible, device-independent gait signature. The resulting gait hash may be used to serve as a primary or secondary credential for biometric identity verification in both clinical and industrial access control applications.

The system may also include a master orchestration engine. This orchestration engine may be used to govern the routing of each patient's data into one or more artificial intelligence components based on one or more of:
the user's registered diseases; comorbidities;
the user's demographics (age, gender, ethnicity);
the user's active medication lists; and
the user's historical gait data baselines.

By performing this function, the orchestration engine creates a fully individualized AI model-to-patient mapping, resulting in a one-to-one adaptive AI clinical relationship. The orchestration engine thus uses each patient's retrieved data to perform routing decisions and, if necessary, activates relevant trained AI models to receive the patient's data. Thus, multiple trained AI models can receive a patient's data in parallel and, as such, multiple analyses of the patient's data can be executed simultaneously.

As another feature of the system, the system incorporates verticalized AI models (“Vertical LLMs”) specifically trained on gait patterns associated with distinct medical conditions. Vertical components cover multiple clinical domains, including but not limited to:
Neurological conditions such as: Parkinson's Disease, Huntington's Disease, Multiple Sclerosis, Alzheimer's Disease, Stroke, Frontal Gait, Gait Apraxia, Spinal Cord Injury, Cerebral Palsy
Orthopedic conditions such as: Osteoarthritis, Hip Dysplasia, Knee Osteoarthritis, Ankle Instability, Scoliosis, Muscular Dystrophy
Metabolic conditions such as: Diabetes, Diabetic Neuropathy, Obesity, Fibromyalgia, Nutritional Sarcopenia
Cardiovascular conditions such as: Congestive Heart Failure, Orthostatic Hypotension, Peripheral Vascular Disease
Geriatric/Aging conditions such as: Frailty, Polypharmacy-Induced Gait Risk

Implementation of the vertical AI models may be accomplished by continuously training and retraining the models for the above conditions to ensure up-to-date models. In one implementation, baseline loops are compared to current loops for users that have known/expected gait impacts due to their conditions. As an example, baseline loops (pre-onset) and current loops (post onset) for multiple users with Parkinson's Disease are used to train vertical AI models. The differences between the baseline and current loops or the actual baseline and current loops are used to train the vertical AI models. Such trained AI models can thus learn to identify effects of conditions on user gaits. The trained AI models can, provided with baseline and current loops or differences between baseline and current loops, classify/identify the effects of a user's condition on their gait. A user with baseline and current loop data can thus be provided with potential condition diagnoses based on whether the trained AI models detect similar/same effects of a condition on the user's gait. Each vertical AI model can thus classify gait anomalies, can predict or determine disease progression, and provides clinical insight based on deviations from patient-specific baselines.

Additionally, the system includes horizontal pharmacological AI components (“Horizontal LLM”) that evaluate potential gait impacts caused by prescribed medications. This component leverages/takes into account:
FDA black box warnings;
Clinical trial datasets;
Pharmacovigilance reporting systems;
Drug-drug interaction databases; and
Drug mechanism-of-action pathways.

Implementation of the horizontal AI models may be accomplished by continuously training and retraining the models for the above considerations to ensure up-to-date models. In one implementation, baseline loops are compared to current loops for users that have known/expected gait impacts due to their medications. The differences between the loops or the actual baseline and current loops are used to train the AI models. Such trained AI models can thus learn to identify effects of treatment regimes on user gaits. The trained AI models can, provided with baseline and current loops or differences between baseline and current loops, classify/identify effects of a user's treatment regime on their gait. Depending on the implementation, the severity of effects of the user's treatment regime on their gait can also be ranked/scored by one or more of the trained AI models. The system detects medication-induced gait abnormalities, cross-drug interaction risks, and calculates drug-gait interaction risk scores.

In terms of medication exposure functions that may be used by the system, the following example is provided. For each medication d and dose event i at administration time τ_{dl}, the system computes a time-varying exposure function

$${Ψ}_{di} ( t ) = {α}_{d} \cdot {f}_{ab s} ( t - {τ}_{di} ) \cdot {e}^{- {λ}_{d} ( t - {τ}_{di} - {δ}_{d} )} \cdot {1}_{t \geq {τ}_{di} + {δ}_{d}} ,$$

where δ_{d} is absorption lag, λ_{d} relates to half-life (t_{1/2}=ln λ/λ_{d}), f_{abs} models formulation-specific absorption (IR/ER), and α_{d} scales expected effect magnitude.

The cumulative exposure is Ψ_{d} (t)=ΣᵢΨ_{di}(t).

Other variables that may be taken into account include patient covariates (age, renal/hepatic function, body mass) adjust α_{d} and λ_{d}. Active metabolites are modeled with additional compartments whose contributions superimpose onto Ψ_{d} (t)

Similarly, gait features (e.g., stride/stance time variability, step asymmetry, center of pressure path length, medio lateral sway, jerk, harmonic ratio, loading rate) may be time aligned to Ψ_{d} (t) to form exposure aligned feature matrices.

The system may also use causal inference. This means that, within subject designs, this may include dechallenge/rechallenge, case crossover (hazard vs. control windows), and interrupted time series with synthetic controls, the system may estimate medication effects separately from disease progression.

Additionally, the system may implement knowledge graph regularized attribution. Thus, the system may implement a pharmacological knowledge graph (drug→class→mechanism→pathway→gait phenotype) that regularizes attribution via graph constrained penalties, yielding a drug-gait attribution vector over agents and interactions.

The system may implement an interaction detection methodology. Accordingly, super additive and sub additive interactions may be detected by contrast tests over paired and higher order terms in the attribution vector. Additionally, the concept of uncertainty may be included in the system. This may take the form of interaction scores and attributions being configured to include uncertainty bounds (bootstrap or Bayesian posteriors) surfaced with clinician readable rationales. As well, the system may implement the idea of counterfactual simulation with safety overlay. For this implementation, a simulator forecasts outcomes under dose timing shifts, IR↔ER swaps, and substitutions; recommendations are gated by black box warnings, contraindications, and clinician rules before display.

For the system's reporting functions, these may be enhanced such that outputs include exposure overlays, feature importances, and mechanism hypotheses sourced from the knowledge graph, each tagged with model/version identifiers.

As another feature of the system, the platform performs continuous, longitudinal gait tracking. As noted above, trained AI models compare newly acquired gait data against historical baselines and thus calculate one or more of:
Disease-specific regression or progression status;
Deviation scores from baseline thresholds; and/or
Stability scoring over time.

For the treatment block, the system may implement composition decomposition methodology. Accordingly, medication records are decomposed into components c∈C_{d}: active ingredient(s), salt/polymorph, release mechanism (matrix/osmotic/multiparticulate), and excipients, plus manufacturer/brand, site, and lot/batch metadata. For each component c, the system computes Ψ_{d,c} (t) parameterized by dissolution/release kinetics; Ψ_{d,c} (t) may be convolved with Ψ_{d} (t) to capture component-specific release profiles. Additionally, the system may provide estimates for a component-level attribution vector allocating observed gait/balance/weight changes to specific components and interactions.

The system may also include subsystems that implement release-mechanism morning instability assessments; sugar-alcohol excipient sway in fasting state; flagged lot anomaly with cross-site confirmation. Additionally, the subsystems may implement:
Hierarchical Pooling. A hierarchical model pools evidence across brands, sites, and lots, with random effects quantifying component- and lot-level contributions.
Lot Signal Detection. Change-point/outlier detection across time and deployments flags lot/batch anomalies, emitting alerts with confidence estimates.
Reformulation Simulator. An R&D simulator varies excipients, salt forms, and release mechanisms (or brand/lot substitution) to forecast outcomes with uncertainty bounds. The simulator may be configured to evaluate not-yet-marketed virtual compositions, aiding pre-market design decisions.

This continuous longitudinal monitoring allows for proactive clinical interventions, early detection of deterioration, and therapy adjustment.

Furthermore, as a further feature, the system generates standardized clinical reports in formats compatible with electronic health record (EHR) systems, including FHIR-compliant structures. Such reports may include any of the following:
Disease classification;
Progression/regression scoring;
Drug-induced gait alerts;
Gait metric trends over time; and/or
Personalized care recommendations.

These reports are automatically generated by the system based on the results of the various analyses. Depending on the implementation, preconfigured template reports may be created with relevant fields and sections being filled in by the analyses results as well as with data gathered by the system from the actual gait-related readings.

To ensure data security, the platform may be configured to operate within a secure cloud-native deployment. Features of such a deployment may include the use of one or more of:
Containerized microservices (e.g., Docker, Kubernetes);
Role-based access control (RBAC);
DevSecOps CI/CD pipeline integration;
Encrypted biometric storage (AWS KMS, Vault); and/or
Zero-trust access models.

The system may also be configured to be compliant with HIPAA, GDPR, and the Biometric Health Identity Act to ensure compatibility and workability with different government/governing authority standards.

In one preferred embodiment, a user is equipped with gait-sensing/gait-data detecting insoles which capture real-time gait parameters. The data is transmitted to cloud-based AI-based processing nodes in one or more servers. These trained AI models are configured to generate/process the gait-based data. The server(s) generate a unique gait hash which may be used for biometric identity verification. As well, the system allows for personalized AI-based orchestration (through an orchestration component) that routes user data to the relevant vertical disease/condition models based on the user's medical history.

Also for this preferred embodiment, the system includes pharmacological AI-based components that evaluate active medications for gait-related side effects. The system would also calculate/determine disease-specific or condition-specific progression scores that are based on current gait data deviations from baseline gait data. As noted above, the system would also automatically create/generate clinician-facing reports for professional review. Such reports would include longitudinal graphs, drug-gait warnings, and risk scoring. Preferably, the user's data is securely exported to the clinic's EHR system. For an enterprise deployment, the gait hash (detailed above for identity determination purposes) may also serve for secure biometric access control in industrial or governmental settings.

The various aspects and embodiments of the present invention provides a number of advantages that are not present with the prior art. Such advantages include: combining biometric identity with clinical AI disease monitoring, fully personalized one-to-one adaptive AI interaction, integrating drug-side-effect analysis directly into gait-based clinical monitoring, longitudinal patient tracking using objective gait data, advantageous use in both clinical and non-clinical (industrial, security) applications, and provides a secure, scalable cloud deployment compliant with healthcare regulations.

In one aspect, the system of the present invention provides a system for biometric identification, chronic disease monitoring, and personalized AI-based clinical analysis with the system comprising: one or more gait data acquisition devices configured to capture gait metrics from a user; a biometric identity engine configured to generate a unique gait signature for user authentication; one or more vertical artificial intelligence components trained to detect and classify disease-specific gait characteristics; one or more horizontal artificial intelligence components configured to evaluate the user's prescribed medications for gait-impacting side effects; an orchestration component configured to dynamically route user data to appropriate AI components based on medical history, demographics, and comorbidities; a longitudinal monitoring component configured to calculate disease progression or regression relative to individualized baseline metrics; and an export component configured to generate clinician-ready reports and synchronize data with external health records.

In this aspect, the orchestration component is used to create a personalized adaptive AI model specific to the user based on one or more of the user's registered health condition(s), medication list, demographics, and previous longitudinal gait history. This is performed to establish a dynamic one-to-one AI interaction for individualized disease monitoring. For clarity, the vertical AI components may include distinct neural networks or language models specialized for one or more of: neurological disorders, orthopedic disorders, metabolic disorders, cardiovascular disorders, autoimmune disorders, or geriatric aging conditions. Similarly, the horizontal AI components may be used to cross-reference prescribed medications against drug databases, clinical trial data, pharmacovigilance literature, and known gait-related side effects to identify drug-induced gait abnormalities.

In another aspect, the orchestration component concurrently invokes multiple vertical and horizontal AI components to evaluate overlapping or co-existing medical conditions for the user. The system may include a progression scoring component that compares new gait data to prior baselines and generates quantifiable regression, progression, or stability scores for each tracked condition. As well, the biometric identity engine may generate a gait hash uniquely derived from multi-dimensional gait parameters that include stride length, cadence, gait speed, variability, balance index, and load-bearing metrics. This gait hash may be used to securely bind wearable devices to authorized users, thereby enabling biometric device pairing and access control.

The system may also be configured such that deployments emit de-identified summary statistics to a secure aggregation service. As well, the use of optional differential privacy would guard against re-identification. The system may also implement the use of disproportionality analysis and empirical-Bayes shrinkage to compute drug/excipient/brand/lot signal scores. The system may allow for the enforcement of data residency/jurisdiction constraints for HIPAA/GDPR/biometric privacy compliance.

In terms of the AI trained models, aggregated signals may be used to update local models as bounded priors.

In another aspect, the orchestration component further incorporates user demographics, ethnicity, weight biomarkers, and comorbidities as multimodal inputs into its AI model routing decisions. Additionally, the user interface may include input components for capturing real-time patient data with the input components being directly coupled to backend orchestration services via secured API endpoints that support encrypted data exchange, session authentication, and patient-specific data mapping.

For clarity, the AI components may be deployed as distributed microservices within a cloud-native infrastructure that supports real-time inference, asynchronous processing, and scalable workload management. In addition, the export component may be configured to format patient reports into standardized electronic health record formats including FHIR (Fast Healthcare Interoperability Resources) standard-compliant structures for clinician interoperability.

In terms of specific implementation details, the gait data acquisition component may be configured to capture one or more of: stride length, cadence, gait speed, stride variability, center of force, balance score, load-bearing weight, standing weight, step count, and weight biomarker shifts. Similarly, the vertical AI components may be configured to analyze disease-specific gait profiles selected from a library of various medical gait conditions. For some implementations, the library may include at least 25 medical gait conditions. Furthermore, the horizontal AI component may include a pharmacological knowledge graph linking medications to mechanisms of action and their respective biomechanical gait impacts. For better results, gait data acquisition, AI inference, and longitudinal monitoring are performed continuously or at defined intervals as these enable proactive real-time detection of clinical deterioration.

The system may be configured to include a clinical alert component that automatically notifies care providers when pre-defined gait or progression thresholds are exceeded. The system may further include a secure role-based access control layer for managing patient data visibility across care teams, administrators, and enterprise clients. This access control layer is preferably configured to be fully compliant with HIPAA, GDPR, and biometric privacy laws. Multiple instances of the system may be deployed across geographically distributed cloud clusters with federated AI orchestration. This configuration would allow for global patient monitoring while preserving local data residency compliance.

Other features of the system may include:
On device models detect orthostatic instability or sedation and can temporarily gate high risk industrial tasks;
Cloud models perform confirmatory causal analysis before finalizing holds or releasing gates.
Edge events record device ID, model version, and timestamps for audit.

The various instances of the system may have AI components that are configured to support post-marketing drug safety surveillance, clinical trial monitoring, and pharmacovigilance signal detection based on population-level gait pattern shifts.

The system of the present invention may be used in many contexts. As examples, the biometric identity engine and the gait analytics platform may be applied to industrial safety credentialing, government security access systems, and chronic disease management programs.

In terms of functionality, the system may be configured so that the orchestration component executes rule-based and AI-based routing logic dynamically at runtime to invoke appropriate disease-specific vertical models and drug interaction horizontal models based on updated patient input, medication changes, or new clinical diagnoses. Additionally, the system may be further configured such that each vertical and horizontal AI component maintains version-controlled model instances. Model updates may be deployed through controlled pipelines with traceable version identifiers and auditable change logs to thereby ensure clinical safety and regulatory compliance. In one implementation, the platform may operate as a multi-tenant cloud-native deployment that isolates enterprise, government, healthcare, and research clients into secure logical partitions with independent data governance, encryption keys, and access control frameworks.

The various aspects of the present invention allow for multiple additional functionalities of the various components. As an example, the orchestration engine may perform feature fusion across multiple vertical AI components. Such functionality would allow for simultaneous classification of overlapping conditions by synthesizing feature vectors drawn from neurology, orthopedic, metabolic, cardiovascular, and aging-specific biomarker profiles. As an additional functionality, the AI orchestration layer may dynamically generate disease-specific prompt templates for each patient based on stored demographic data, historical progression markers, medication lists, and comorbidities. The effect of this additional functionality is that the vertical AI components receive individualized prompt inputs for every inference task.

Additional features of the system may include EHR Interoperability. For this, reports and alerts may be exported as FHIR resources including Observation (gait metrics), Detected Issue (drug-gait risks), and Medication Statement/Medication Request mappings for medication context.

According to another aspect of the present invention, the system may be configured for AI safety. This may be accomplished by having the AI inference outputs processed through an AI safety overlay component. The safety overlay component may be configured to apply constitutional AI filters, clinical boundary conditions, and ethical safety constraints before any diagnostic or therapeutic recommendation is presented to a clinician or patient.

As another feature of the system, the system may be configured to address the potential issue of AI model drift. This can be done by configuring the system such that the AI components include automated model drift detection and alert systems. These drift detection and alert systems may be configured to monitor prediction stability over time and to trigger administrative review upon detection of statistically significant deviations from prior baseline inference accuracy.

Much like the other features noted above, the system may be configured to ensure secure functionality. This may be implemented by ensuring that vertical and horizontal AI components are deployed across geographically distributed federated learning clusters, thereby allowing for localized model fine-tuning while preserving data privacy, minimizing cross-border data transfer, and supporting jurisdictional compliance.

The system, in a further configuration, allows for personalized alerts. This may be implemented by having each patient's personalized alert thresholds for disease progression be adjusted automatically by the AI engine based on that patient's unique combination of gait history, medication effects, comorbid conditions, demographic risk factors, and prior response trends to therapeutic interventions.

In addition to the above features and components of the system, the system of the present invention may include an insurance modeling block that is configured to ingest gait-based disease progression data and calculate dynamic risk scores. These risk scores may be used for continuous underwriting of health or life insurance coverage for the user. This insurance block may also be configured to automatically adjusts insurance premiums based on real-time analysis of the user's condition-specific gait regression, stability, or improvement, as determined by the trained AI models. Similarly, the insurance block may also be configured to trigger predefined policy actions including claim holds, risk tier reclassification, or care navigation alerts when AI-derived gait metrics exceed preset clinical or functional risk thresholds. As an added feature, the insurance block may be configured to incorporate outputs from the pharmacological AI block. This incorporation allows for gait impairments attributed to drug side effects to be factored into insurance underwriting decisions, premium adjustments, or policyholder risk classification.

Furthermore, the system may include one or more AI engines that are configured to continuously generate and store longitudinal health performance data suitable for actuarial analysis, insurance product pricing, and portfolio-level risk stratification across populations. The various AI models and their outputs (including biometric gait identity and disease progression data) may be jointly analyzed to detect inconsistencies or anomalies indicative of potential insurance fraud or falsified impairment claims.

Referring to FIG. 8C, illustrated is a block diagram detailing the data connections between the various blocks and components of an AI-centric system according to one aspect of the present invention. As can be seen, the system 1100 includes a server 1110 that receives gait data from a wearable data gathering device 1120. As noted above, the device 1120 can be a wearable insole with multiple sensors. The server 1110 includes an identity hash block 1130, an orchestration component/block 1140, a vertical AI model block 1150 (diagnosis block), a horizontal AI model block 1160 (treatment block), and a report generation block 1170. As can also be seen, the server 1110 includes an internal database 1180 for storing previously generated/previously gathered user data. The system 1100 may also access/use an external database 1180A

As explained above, the identity hash block 1130 receives gait data from the device 1120 and generates/calculates a hash from this data. The resulting identity hash will be unique to the user based on that user's gait data. The identity hash is useful for determining the user's identity and may be used to unlock the user's data. The identity hash may also be used to verify the user's identity so that the user can be provided physical access or virtual access to locked down/segregated resources (e.g. data, access through doors, or physical access to locations). In the event the user's gait changes or is affected by the user's condition(s), the system may be configured to use the immediately previously gathered data for the next baseline hash for the identity hash. This ensures that, even as the user's gait drifts or changes as time goes on, the user's identity hash is still useful. As an example, data gathered at time t[x−1] is used to generate the identity hash (base) and, at time t[x], the data gathered at t[x] is used to generate an identity hash (current). The base hash is then compared to the current hash to confirm user identity. The current identity hash is then used as the base hash for the next time the user identity needs to be verified. Thus, base hash at time t[x−1] is the current hash at time [x−2]. Similarly, the current hash at time t[x−1] is the base hash for time t[x] while the current hash at time t[x] becomes the base hash for time t[x+1]. This ensures that, even as the user's gait changes due to the user's condition(s), such drift is taken into account for the identity hash function of the system.

Accordingly, the system may thus use a rolling baseline for its identity hash. Thus, to maintain authentication despite clinical drift, the system updates the baseline gait hash using the most recent confirmed session. A current hash Hₜ is compared to a prior baseline Hₜ₋₁ for verification; upon success, Hₜ becomes the next baseline. Rolling updates preserve device-independent identity while tolerating longitudinal gait changes.

For the orchestration block 1140, this block receives the identity hash from the identity hash block 1130 to determine the user's identity. In addition, the orchestration block 1140 receives the current data from the device 1120. The orchestration block 1140 then retrieves the user's data from the database 1170 and, based on this data, the orchestration block 1140 routes the current user data to specific trained AI models in the diagnosis block and in the treatment block. For clarity, the orchestration block retrieves user data from the database and, based on which conditions the user may have or which conditions the user may be diagnosed for, the orchestration block sends the user data to these trained AI model components in the diagnosis block. Similarly, based on the retrieved use data, the orchestration block determines which treatment regimes the user may be under and, accordingly, sends the user data to the corresponding trained AI model components in the treatment block.

Other features of the system may include:
Immutable Data Products. Each schema defined transform emits an immutable, content addressable object identified by a cryptographic digest; objects reference their parent to form a transform DAG;
Identity at the Edge. Identity is computed at the edge (e.g., gait derived) to authenticate the data source; downstream data products do not embed identity;
Identity Gating. An identity gating service issues scoped, revocable tokens authorizing permitted joins/transforms without revealing persistent identifiers;
Deterministic Execution. Transforms and ML run in deterministic containers pinned by image digest, dependency locks, and fixed seeds, ensuring bit for bit reproducibility;
Model Registry. A registry stores model artifacts, code hashes, hyperparameters, input data product digests, and environment fingerprints; promotion gates require drift validation and reproducible retraining on golden datasets;
Transform Level Lineage. For each data product, lineage metadata include input digests, transform IDs, operator identity (tokenized), timestamps, and environment fingerprints;
Explainability Binding. Explainability artifacts (feature attributions, exposure overlays) are bound to model/data product digests;
Identity Safe N: N Evaluation. Datasets in separate token scopes may be compared with salted, scope restricted comparators that produce matches/metrics without exposing identifiers;
Compliance Posture. The immutable DAG, deterministic environments, and registry gates yield predictable versioning aligned with healthcare regulatory audits;
Content Addressing. Content digests include payload bytes and schema version to prevent silent mutation of prior states;
Revocation. Token revocation terminates future operations without re writing existing objects.

Referring to the vertical AI model block 1150 (the diagnosis block), FIG. 8D is provided. As can be seen, the orchestration block 1140 is connected to multiple trained AI models 1150A-150D. Each of the trained AI models 1150A-1150D in the diagnosis block 1150 corresponds to a different condition/disease/ailment that a user may have. Each of the models 1150A-1150D has been trained to assess the progression or regression of the condition/disease based on a user's gait data. The user's gait data may include the baseline gait data, the current gait data, or simply the differences between the baseline and current gait data. As noted above, the different models are trained with datasets from users with the condition and, by showing the differences between the baseline and the current data sets in the training datasets, the models are trained to detect/assess the progression or regression of the condition/ailment. In addition, each of the models 1150A-1150D may be configured to assign a score or a rank to the progression or regression of the condition/ailment in the user.

As a variant, the models 1150A-1150D may also be used to determine if a user has the condition or disease for which the model is trained. As an example, the user's current gait data and baseline gait data are sent to one of the AI trained models for, as an example, Parkinson's disease. Based on the differences in the gait data, the model can provide an indication as to whether the user has or may have Parkinson's disease. Of course, any conclusions that an AI trained model may have regarding a user's diagnosis or condition may be subject to review by a medical professional.

For the horizontal AI model block 1160 (treatment block), the function is similar to the vertical AI model block 1150 (diagnosis block). The treatment block 1160 contains AI trained model blocks 1160A-1160D that are configured to receive the user's gait data. Based on the trained AI model's assessment of the gait data, each model generates an effectiveness of a treatment regime on the user's overall condition. Similarly, each trained AI model 1160A-1160D is configured to assess any side effects that the treatment regime may have introduced into the user. As an example, a trained AI model 1160A may correspond to a specific pharmaceutical treatment regime (e.g. take medication X at dosage Y once a day every other day). The trained AI model 1160A can then determine if the treatment regime has any notable effects (side effects or otherwise) on the user's gait. Each trained AI model 1160A-1160D corresponds to a treatment regime (pharmaceutical or otherwise) that a user may be subject to and the orchestration block determines which treatment regimes are being used with a particular user. The relevant AI trained models corresponding to the treatment regimes for a specific user are then activated and the orchestration block sends the user's data to these models. Each model then generates data that indicates the gait-related effects (if any) of its corresponding treatment regime on the user. The output of each trained AI model can then be used to generate reports relating to the user's condition.

It should be clear that the orchestration block determines the AI models to send the data to in both the treatment and diagnosis blocks concurrently. As well, the user data is sent to these AI models and that the treatment and diagnosis assessments and analyses by the various trained AI models occur simultaneously.

In addition to these blocks, the system may include a report generation block 1170 and a database 1180. As can be imagined, the report generation block 1170 receives the output of the various trained AI models from both the treatment block and the diagnosis block. The outputs of these trained AI models are then used to automatically generate reports relating to the user's condition, the progression or regression of any conditions, as well as the effects of any treatment regimes the user may be under. The automated generation of reports may be accomplished through the use of preconfigured templates where the fields relating to the outputs of the specific trained AI models are populated from the results of the treatment block and of the diagnosis block. These reports can then be sent outside the system for review/assessment by medical professionals 1190.

For clarity, the database 1180 may be part of the server or may be external to the server. Additional databases may also be accessible by the server depending on the implementation. These databases may be used to store user data including user identity hashes, user condition records, user treatment regimes, as well as old user condition reports.

As another variant, the system 1100 may include an insurance block 1190. As can be seen from FIG. 8F, the insurance block 1190 receives condition progression or condition regression data from the diagnostic block. The block 1190 includes a score calculation component 1200 that calculates dynamic risk scores based on the progression data from the diagnostic block. These risk scores can be used for continuous underwriting of health or life insurance coverage for the user. Additionally, the insurance block 1190 may include an adjustment component 1210 that also receives the progression/regression data from the diagnostic block. The adjustment component determines if the user's insurance premiums need to be adjusted based on the results from the data received from the diagnostic block. As an example, if the disease/condition is progressing, then the user's premiums may need to be increased while if the disease/condition is regressing, then the user's premiums may be lowered.

As a further variant of the insurance block 1190, a trigger component 1220 may be included. The insurance block 1190 also receives the user's gait data (including baseline/previous gait data and current gait data) and, based on the gait data, the trigger component determines if predefined policy actions (e.g., policy holds, risk tier reclassification, care navigation alerts) need to be triggered based on the user's gait data. The triggering of events/policy actions may occur when the user's gait data indicates an event that requires the policy action. As an example, the gait data may indicate that the user's mobility is severely impaired when, previously, the user's mobility was only slightly impaired. Events (as indicated by the gait data) such as the exceeding of preset clinical or functional risk thresholds may be used to trigger specific policy actions. Of course, the type, frequency, or specific identity of the event may be programmed and determined based on implementation.

As another variant, the insurance block may be configured to receive output data from the treatment block. The effects of treatment regimes on user gait may be used in determining insurance underwriting decisions, premium adjustments, or policy risk classification. Such measures would ensure a more complete picture of the user's condition and situation in light of any effects that treatment regimes may have on the user.

The insurance block may also include a subsystem that provides payer/actuarial outputs. This may be a mobility-aware component that computes risk and formulary rankings conditioned on component-level attributions and costs.

Other features of the system may include the use of:
Exposure Function/Kernel: Time-domain weighting mapping dose events to expected effect on gait features.
Component-Level Attribution Vector: Allocation of observed outcome changes across composition components.
Content-Addressable Data-Product: Immutable, schema-defined object identified by a cryptographic digest.
Transform DAG: Directed acyclic graph of data-products linked by parent pointers.
Identity-Gating Token: Scoped, revocable authorization for operations without persisting identity.
Golden Dataset: Immutable dataset snapshot used for regression and reproducibility testing.
Device-Agnostic Processing. Analytics, orchestration, and safety functions disclosed herein operate independently of the specific device class, provided the normalized input schema is satisfied.

As noted above, the system is interoperable with or may be used with:
any body-worn or proximally carried sensor platform including insoles, shoes, hearables (earbuds/eartips), seeables (smart glasses/visors), wrist-worn devices (watches/bands), chest/torso patches, belt/garment sensors, and medical-grade devices (including defibrillators/ICDs, pacemakers, ECG/PPG/SpO₂/BP/respiration monitors), whether consumer or clinical grade;
any device certified for clinical use that streams physiologic telemetry (e.g., defibrillator/ICD, ECG patch, Holter, ambulatory BP monitor) to the system.

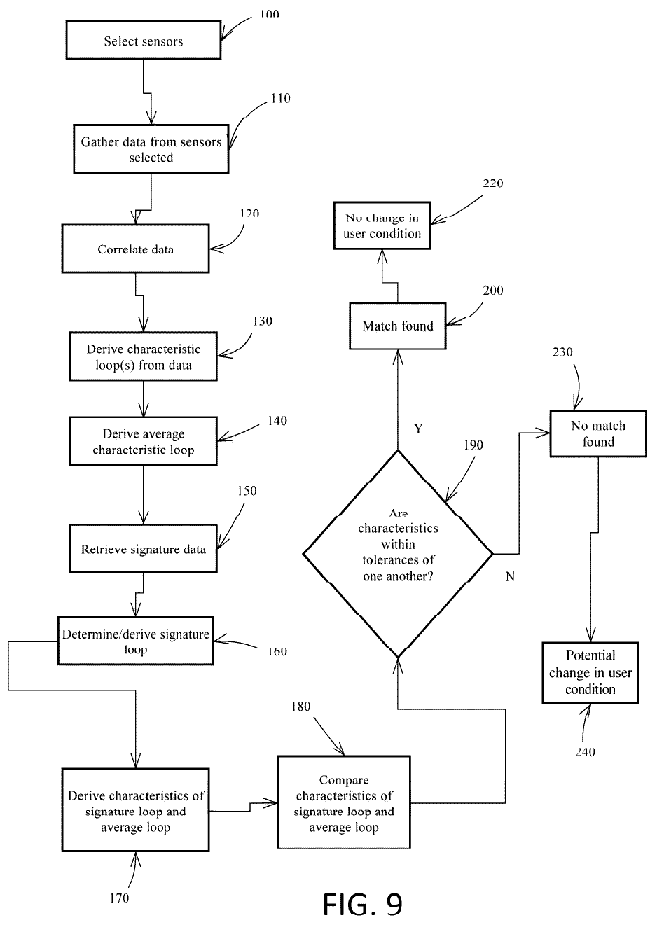

Referring to FIG. 9, a flowchart of the process described above is illustrated. To store the signature data into the system, the initial step 100 in the process is that of selecting two of the sensors to be used in the comparison process. As noted above, the sensors are, in one embodiment, inserted or installed in a user's shoe. Once the sensors have been selected, data is gathered from these sensors as the user walks normally (step 110). Once gathered from the sensors, the data is then correlated with one another to form the data pairs noted above (step 120). This means that data points from one sensor is mated with data points from another sensor. With the data pairs in hand, at least one characteristic loop can then be created/derived from the data pairs (step 130). Depending on the configuration, discrete steps may be separated from one another so that each step may have its own characteristic loop. Alternatively, an average characteristic loop may be derived from the data values from the sensors. Once the average characteristic loop has been found (step 140), the signature data can be retrieved (step 150). The signature data, depending on configuration can then be used to determine the signature loop (step 160). The characteristics of both the average characteristic loop and the signature loop can then be calculated or derived from the two sets of data (step 170). The characteristics are then compared (step 180), taking into consideration the preprogrammed tolerances. If the characteristics from the two sets of data are the same (step 190) (within the preprogrammed tolerances) then a match is found (step 200) indicating no change in the user's condition (step 210). If they are not within the preprogrammed tolerances, then no match is found (step 220) and a potential change in the user's condition is indicated (step 230). The indication of whether a match is found or not found can then be communicated with the server 50. The server 50 may then communicate with the distributed databases to gather data and/or correlations between the user's characteristic data and potential/actual medical and/or physical conditions.

The process may also be seen as eight specific steps:

The first processing step after retrieving the data is one where the pair sensor signals are filtered applying DFT (Discrete Fourier Transform) based low-pass filter. The cut-off frequency of the filter is defined taking into account a Nyquist frequency (related to the sampling rate) on the high end, and a main signal frequency (related to the walking speed of the individual) on the low end. Walking frequency estimation is also a part of the described processing step.

Using an FFT (Fast Fourier Transform) implementation technique and sync-filter as a benchmark, a low pass filter with flat pass-band (low ripple) high stop band attenuation may be used. Additional advantage is taken from the use of non-causal filters since the hard-real-time processing is not required (signals are registered first and then filters are applied).

The second processing step is a construction of the characteristic loop for the chosen pair of signals. The characteristic loop is an ordered set of points with coordinates (X(i), Y(i)) where X(i) is a first chosen signal and Y(i) is a second chosen signal, i is an index corresponding to the sample number.

An autonomous loop is constructed for the time period (subset of all samples) corresponding to the evolution of both signals from low level to maturity level and back to low level. Such a construction is possible since the low level of all signals have a non-empty intersection corresponding to the foot not contacting the ground.

Due to quasi-periodicity of all signals resulting from the nature of human walking, characteristic loops can be constructed autonomously for several periods in time. Although initially defined for raw signals, autonomous loops can then be constructed for smoothed signals (obtained after the first step processing described above).

The third processing step is that of averaging the loops. Several loops are constructed according to the recording of several steps while the person is walking. Those steps and respectively those loops are subject to significant variations. It has been found that only the average loop provides a stable and robust characteristic of human walking.

Averaging of the loops is done by artificially synchronizing several loops (as corresponding to several steps) followed by weighted averaging of the synchronized loops. Weight factors are computed according to the phase shifts from an estimated reference signal (main walking frequency—as per first processing step).

The fourth processing step consists of extracting initial geometrical parameters from the average loop such as loop length, loop width, direction of longitudinal axes, loop directionality (clockwise or counter-clockwise) and the area inside the loop. Other characteristics/parameters which can be used are the variance of each parameter listed above as computed for individual walking steps and as compared to the average value (computed from average loop).

Other parameters which can be extracted may use:
Geometrical method-identify a point on the loop farthest from the origin (let us call it M) this point is further used to find the length (|OM|) and direction of the longitudinal axis (OM), the width is defined as maximal projection onto the line perpendicular to OM
Statistical approach—considering the loop as the cloud of points, the elliptical fit (correlation analysis) can be applied followed by extraction of the parameters of the fitted ellipse (major and minor axis length and orientation).

Regarding loop directionality, the directionality of the loop is related to the phase shift between signal Y and signal X. Namely, the loop is clockwise if Y signal grows from low level to maturity first, followed by the growth of X signal.

The fifth processing step consists of analysing special cases. It is worth noticing that in some cases, for some pairs of signals, the construction of the loop as described above might yield less than perfect results. This may result in a “degenerated loop” due to a high correlation between signals. The “loop” in such case is located very close to the diagonal. For this case only the point farthest from the origin is actually computed (corresponding to maximal amplitude of both signals).

The sixth processing step consists of comparing the loops computed from 2 separately recorded data. It has been found that the high discrimination efficiency of the proposed parametric representation of the pair-wise average loops (see FIG. 8A as an example). Namely, for several pairs of signals/sensors extracted from the set of 8 signals/sensors, the average loops constructed from the smoothed signals stably demonstrate significant similarities when constructed from the data corresponding to the same individual as well as significant differences from average loops constructed for different individuals.

The seventh processing step consists of combining the results of the comparison of several (up to all 56 possible pairs from 8 different sensors/signals) pairs in order to produce a highly efficient discriminate function. Results from various pairs are first weighted according to the number of parameters that can be robustly estimated to support the comparison of the loops. Finally, the results from various pairs can be fused using Dempster-Shaefer framework for an estimation of the likelihood that loops from the baseline data and the gathered data are similar or not.

In addition to the above processing steps, it should be noted that, for mobility-impaired-based applications, the data gathered can be expected to have a number of behaviours. The characteristics that are extracted when performing the loop signature computation (see above) can be divided in three classes:
A) (Class-1) Dimensionless parameters such as:
(1) loop directionality,
(2) direction of longitudinal axes of the loop,
(3) loop elongation (e.g. major to minor axis ratio), etc., as well as standard deviations of those parameters computed over all of the collected data.
B) (Class-2) Size-type parameters having a single dimension such as:
(1) loop length,
(2) loop width,
(3,4) major and minor axis of elliptical approximation of the loop (see above), etc. as well as standard deviations of those parameters computed over all of the collected data.
C) (Class-3) Area-type parameters having two dimensions such as:
(1) area of the loop,
(2) product of major and minor axis of elliptical approximation of the loop and variance of those parameters computed over all of the collected steps.

These 3 classes of the parameters can be used in different ways in determining the estimation of differences between data sets. For processing the data sets based on mobility impairments, stride length, stride to stride variability, cadence, and other movement data can affect class 1 and 2 data sets while weight, posture and other similar physiological changes can affect class 3 parameters.

As an example of how physical changes can affect the characteristics of the derived loops, one can look at the effects of weight-based differences. For such differences, dimensionless parameters (Class-1) are expected to be invariant to weight changes. Size-type parameters (Class-2) are expected to be proportional to the weight change reflecting the fact that the loop is stretched or contracted according to the weight change factor (i.e. the ratio of newly estimated weight to the older one). Area-type parameters (Class-3) are expected to be proportional to the square of the weight change factor.

For processing of weight change-based data, the processing steps can be summarized as:
1. Extraction of the data pairs that provide the robust estimation of relevant parameters;
2. Estimation of the weight change factor from the class-2 (direct) and class-3 (as square root) parameters and verification of invariance of class-1 parameters;
3. Determination of the hypothetical (average) value of the weight or physical characteristic factor;
4. Analysis of the result based on Dempster-Shaefer framework in order to estimate the likelihood that the gathered data supports the determined value.

Of course, the above steps can also be used to process data to determine changes in gait-based data due to other physiological changes. In step 2, instead of estimating the weight change factor, the change factor due to the physiological change can be performed and verifying that some other parameters are invariant.

It should also be noted that a data histogram of daily loop signatures can be stored in the storage block and can be periodically re-correlated to form a new biometric loop signature which reflects the user's weight gain or loss.

The system described above may be used in any number of ways. The system can be used to determine a user's physical or medical condition as well as whether a fitness program or treatment regimen is effective or not. As is well known, for some physical and medical conditions, the progression of the condition affects a person's gait. Similarly, the regression of the condition also affects the person's gait. As such, by comparing a user's baseline gait data with subsequently gathered gait data, the user's condition can be monitored. If no change in the user's gait is detected, then the physical or medical condition has neither progressed nor regressed. If there is a noticeable change in the user's gait (as evidenced by differences in the loops derived from the baseline gait data and the subsequent gait data) this may indicate progression, regression, efficacy of a fitness program or treatment regimen, or any number of health changes in the user. This determination can additionally be performed by the server, perhaps in conjunction with input from the various databases noted above. As well, the determination may be performed after human verification and checking of the data provided. This determination may be made in conjunction with other clinical tests so as to determine correlation between the loop differences, the types of differences, the amount of the difference, and the different conditions and changes in the condition.

In another embodiment, all of the data processed by the data processing block may be internally encrypted so that external systems would not be privy to the raw data transferred between the sensor component and the data processing block. Prior to transmitting the raw data from the sensor component to the data processing block, the data may be automatically encrypted. As can be understood, the data processing block may be physically remote from the sensor component and, as such, the data transmissions between these components may be vulnerable to the outside. In another embodiment, the data processing block is contained within the insole to ensure that any data transfers between the components are slightly more secure.

In another embodiment, any data transfers or communications between the system and any outside server or network systems are encrypted, preferably with one time encryption schemes, to ensure that outsiders are not able to intercept any usable data. Such precautions would preserve the system user's privacy.

The system of the invention may be used to periodically determine if a user's physical or medical condition is progressing or regressing. As well, it may be used to determine if any fitness program or treatment regimen to which the user is being subjected to has had an effect on the user or on the user's gait. The user's baseline gait data may be gathered when the user first visits a facility properly equipped with the system of the invention. Subsequent visits by the user would entail gathering subsequent gait data sets. The loops derived from the baseline gait data set and the subsequent gait data sets can be compared with one another to view any variances between the user's gait data. The amount of change in the loop characteristics from the different data sets can provide an indication as to the degree of change in the user's condition. Large changes in the loop characteristics may indicate an acceleration in the user's condition and may also indicate whether the user's fitness program or treatment regimen (which may include pharmacological treatments) is effective or not.

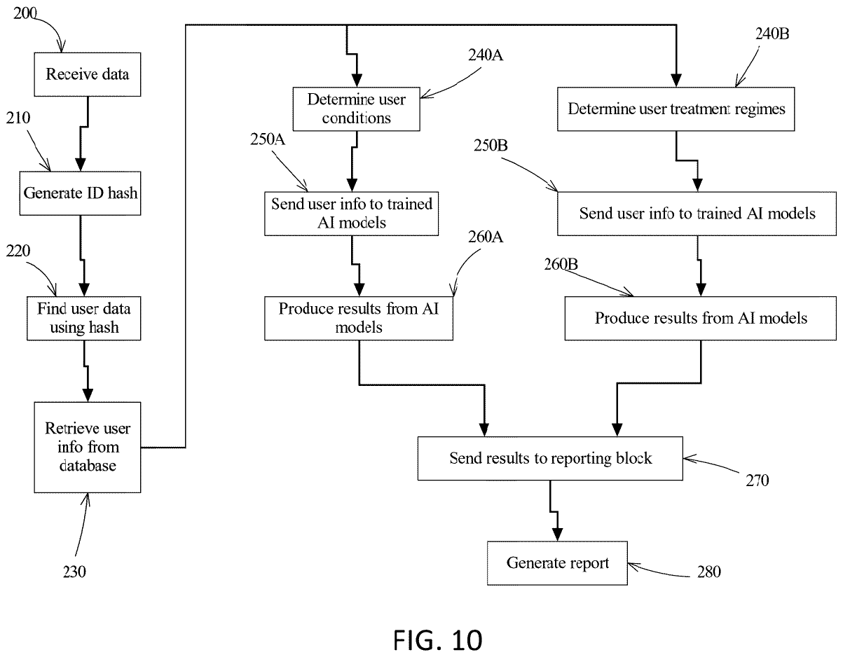

Referring to FIG. 10, a flowchart of the steps executed by the AI-based system of the present invention is illustrated. As can be imagined, the necessary loops and data are generated according to the above description and the AI based steps detailed below are then executed. The comparison/assessment of the similarities/differences/changes between the baseline/characteristic loops and the current loops may be performed by the various trained AI models as detailed above and as will be discussed below. As can be seen from FIG. 10, the steps begin at step 200, that of receiving the current data. The current data may be raw data from the data gathering device or may be the relevant loops as detailed above. The data is then used to generate an ID hash (step 210). The hash is then used to determine the user's identity (step 220) and can be used to retrieve the relevant user's data from the database (step 230).

Once the user data has been retrieved, steps 240A-260A and steps 240B-260B may be executed concurrently. In step 240A, the system determines the user's conditions/ailments. The user's gait data (both baseline/previous and current) are then sent to the relevant AI trained models in the diagnosis block (step 250A). The relevant AI trained models then produce their outputs relating to the user's condition/ailment including progression/regression and/or diagnosis (step 260A). Concurrent with this, the system determines the user's treatment regimes (step 240B). Based on the treatment regimes, the system then sends the user's data (both baseline/previous and current data sets) to the relevant trained AI models in the treatment block (step 250B). The trained AI models in the treatment block then produce their results regarding the effects of the treatment regimes on the user (step 260B).

Once the various trained AI models have produced their results, all the results are then sent to the report generation block to automatically produce reports (step 270). The automated reports are then transmitted to medical professionals (step 280) for assessment/review/action.

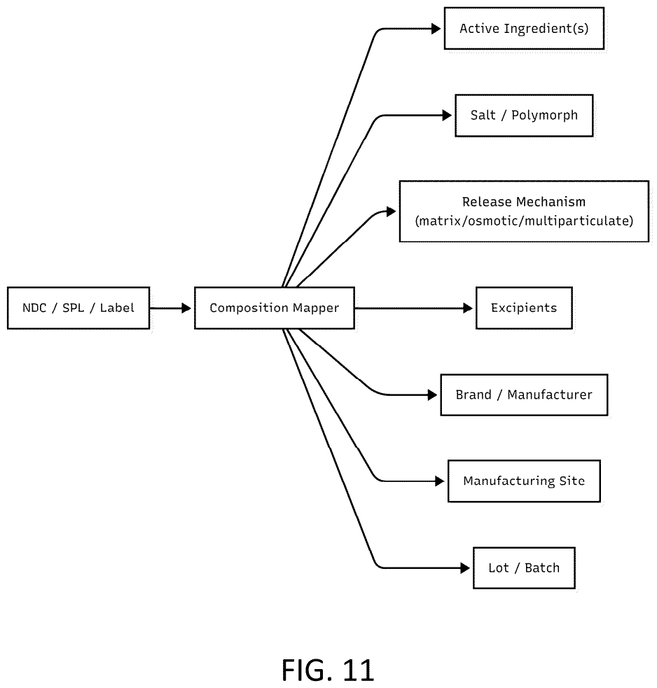

Referring to FIGS. 11-21, illustrated are diagrams of various features and abilities of the system.

In FIG. 11, illustrated is a block diagram of a pipeline that maps product identifiers to active ingredients, salts/polymorphs, mechanisms, excipients, brands/manufacturers and to lots/batches. As can be seen, National Drug Code (NDC)/Structured Product Labeling (SPL)/label data for a product is fed into a composition mapper block and the mapper block maps each product to a whole range of identifying data. Thus, a product can be mapped to its active ingredients, to its mechanism of use/function, its excipients, etc., etc. Accordingly, once a product has been mapped, any reference to that product can easily lead back to its identifiers such as its active ingredients, its brand/manufacturer, etc., etc.

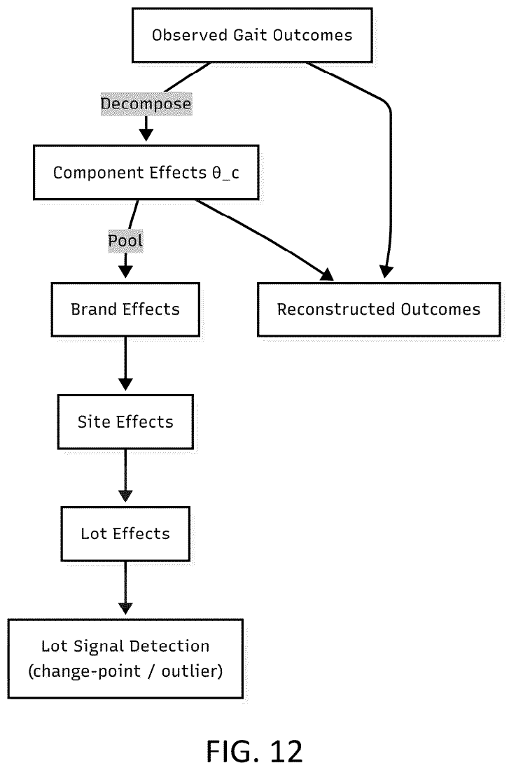

Referring to FIG. 12, illustrated is a block diagram of a hierarchical model that gathers and collates data/evidence across different components, brants, sites, and lots to attribute effects to components, lots, and/or sites. As can be seen, observed gait outcomes are mapped to a decomposition of the treatment regime and the effects of the compositions of the treatment regime. The effects of specific brands, sites, and lots can then be collated/inferred based on the decomposition and the collation of data.

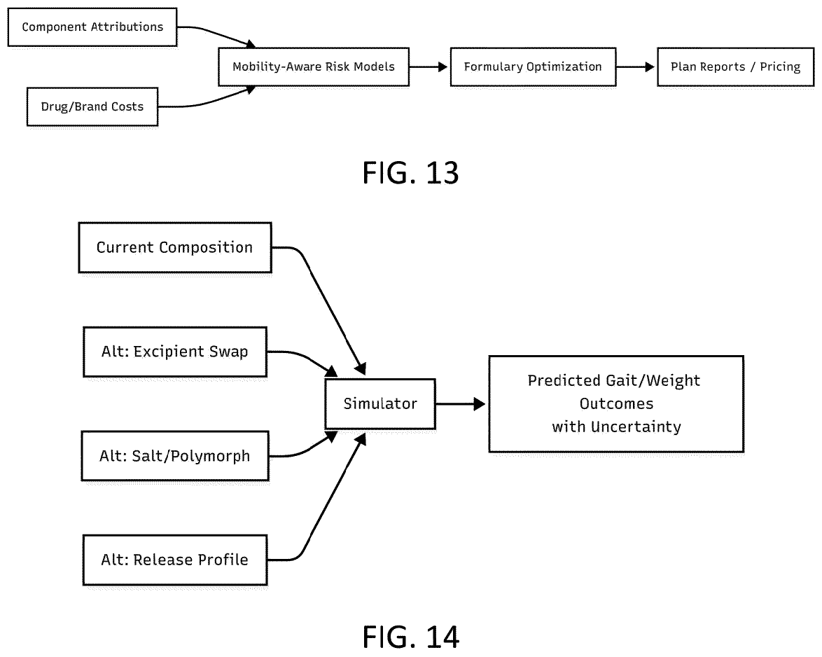

Referring to FIG. 13, illustrated is a block diagram of a dashboard that takes advantage of the results from the model from FIGS. 11 and 12. As can be seen, component attribution and drug/brand costs are input to a risk model block that determines risks based on the components and costs of specific drugs or brands. The risks are then balanced and a formula is optimized (using the optimization block) and reports detailing optimized medication regimes (based on effects/components) with optimized pricing are produced.

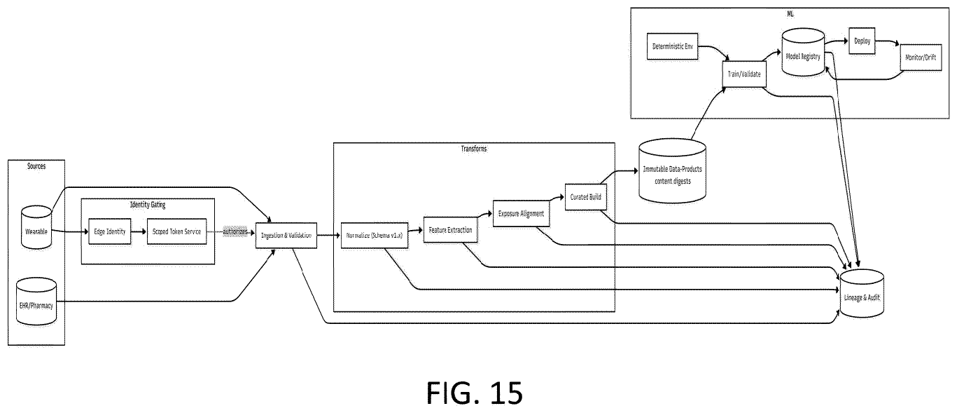

Referring to FIG. 14, illustrated is a block diagram of an R&D reformulation simulator that varies component selections and release profiles. This allows for the forecasting of gait and weight outcomes with uncertainty bonds. As can be seen from the diagram, a current composition fed, along with potential alternatives for salts/polymorphs, potential excipients, and potential release profiles into a simulator. The simulator receives these inputs and outputs predicted gaits or weight outcomes. The simulator therefore takes a current composition and predicts the effects on gait and weight if alternatives were used in the composition. Of course, the predicted outcomes may be provided with uncertainty bounds.

Referring to FIG. 15, illustrated is an identity-decoupled end-to-end pipeline that proceeds from ingestion to identity gating, immutable data-products, machine learning staging, and audit. As can be seen, at the leftmost end are the data sources (e.g., the wearables, the treatment regime sources). The wearable data is passed through and edge identity determination block that ensures that the wearable is worn by someone properly identified. Treatment regime data are ingested along with user data (once the user has been authenticated by the identity determination block) and both are validated. The data are then transformed by passing through normalization, feature extraction, exposure alignment, and a curated build. The outputs of each of these blocks are sent to an audit block. The output of the train of these blocks are sent to a block that digests the results. From the digests, a machine learning block receives the output and validates/trains the relevant models.

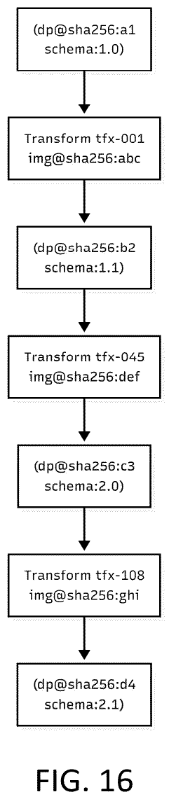

Referring to FIG. 16, illustrated is an immutable transform directed acyclic graph where each schema-defined transform produces a content addressable object. The object includes a cryptographic digest and a pointer to its parent.

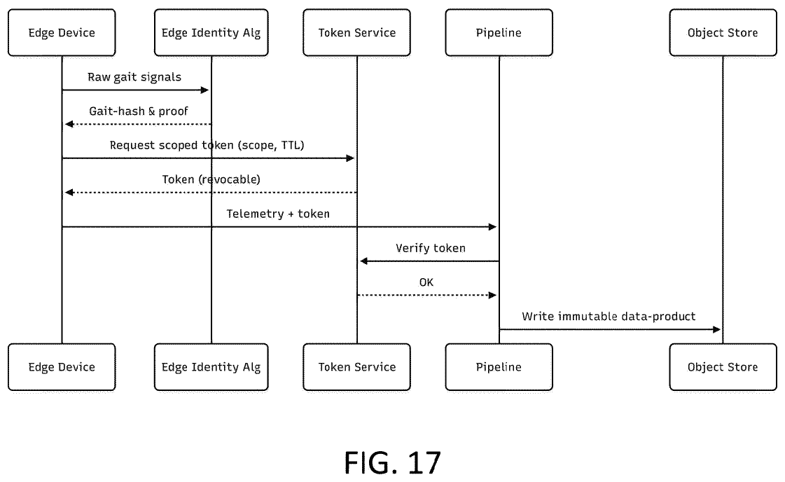

Referring to FIG. 17, illustrated is a block diagram of a sequence that shows edge-computed identity and scoped token issuance. The computed identity and the issued tokens are for used downstream operations. As can be seen, none of these operations involve embedding an identity in the data products. From the figures, it can be seen that the gait signals are sent to an edge identity block that verifies the identity. Once identity has been verified/proofed, tokens are requested and once issued, the tokens are used to travel with the relevant telemetry. The verified token (which is verified before the telemetry is placed in the pipeline) allows for the telemetry is used. Thus, the use of the token allows for an identity verification and use of the data without using the identity with the telemetry/data.

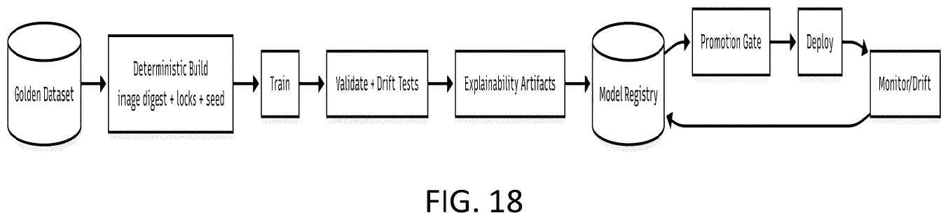

Referring to FIG. 18, a block diagram of a deterministic machine learning lifecycle is illustrated.

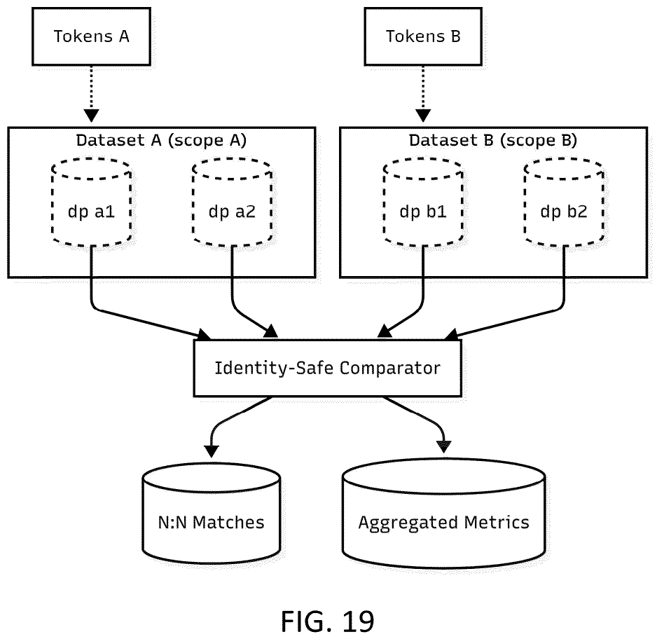

Referring to FIG. 19, shown is a block diagram of a scheme that allows for a many-to-many evaluation without using user identities. The scheme in FIG. 17 uses tokens and these issued tokens are also usable in the scheme in FIG. 19. As can be seen, the issued tokens for users are used to ensure that the data are from verified/legitimate users. The various datasets are then collated without having to use user identities as users are only allowed to have data in the datasets if they have an issued token. Once the datasets are collated, a comparator receives the datasets and the matches and metrics are then gathered from the comparator outputs.

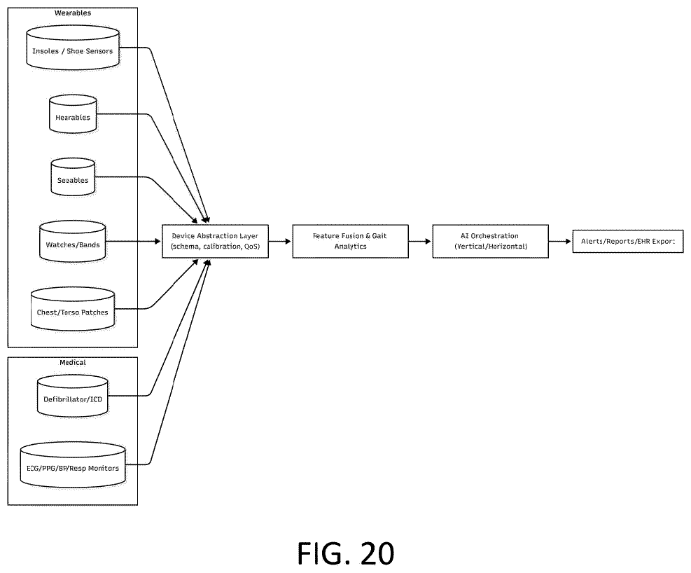

Referring to FIG. 20, illustrated is a block diagram of a multi-device ingestion architecture. As can be seen, various wearable devices and medical devices (including insoles, hearables, seeables, watches, torso patches, and medical-grade devices (including defibrillator/ICD)) stream data to an abstraction layer. The abstraction layer receives the data stream and normalizes the data (e.g., based on schema, calibration, quality of service). The output is normalized data that is then processed for feature fusion and gait analysis. AI orchestration (both vertical and horizontal or for both treatment and diagnosis blocks) is then performed and the results are then use for alerts and reports.

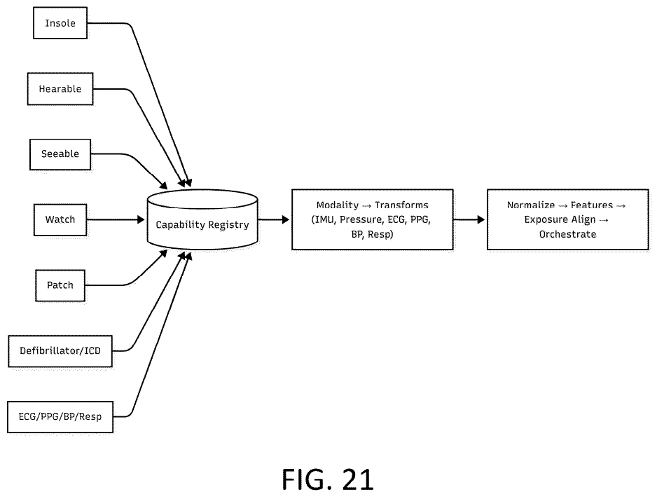

Referring to FIG. 21, is a block diagram for a device abstraction and capability registry that maps device classes to supported modalities and to transforms. As can be seen from the figure, the leftmost blocks are the variable wearable devices. The various devices are registered along with their capabilities. Based on the capabilities of each device, the data from each device is transformed to ensure that the data is actually usable by the system. Once the data has been transformed, then the last block uses that transformed data. The use may be one or more of feature extraction, exposure alignment, and orchestration.

It should be noted that any useful data processing means may be used with the invention. As such, ASICs, FPGAs, general purpose CPUs, and other data processing devices may be used, either as dedicated processors for the calculations or as general purpose processors for a device incorporating the invention.

The method steps of the invention may be embodied in sets of executable machine code stored in a variety of formats such as object code or source code. Such code is described generically herein as programming code, or a computer program for simplification. Clearly, the executable machine code may be integrated with the code of other programs, implemented as subroutines, by external program calls or by other techniques as known in the art.

The embodiments of the invention may be executed by a computer processor or similar device programmed in the manner of method steps, or may be executed by an electronic system which is provided with means for executing these steps. Similarly, an electronic memory means such computer diskettes, CD-Roms, Random Access Memory (RAM), Read Only Memory (ROM) or similar computer software storage media known in the art, may be programmed to execute such method steps. As well, in another embodiment, electronic signals representing these method steps may also be transmitted via a communication network to a network of distributed databases for individual, group or population based analytics by characteristic definition.

Embodiments of the invention may be implemented in any conventional computer programming language. For example, preferred embodiments may be implemented in a procedural programming language (e.g. “C”) or an object oriented language (e.g. “C++”). Alternative embodiments of the invention may be implemented as preprogrammed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink wrapped software), preloaded with a computer system (e.g., on system ROM or fixed disk), or distributed from a server over the network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

What is claimed is:

1. A system for assessing a user's conditions, the system comprising:
a wearable device comprising a plurality of sensors producing data readings;
a server configured to:
receive said data readings from said wearable device;
combine and organize said data readings into specific groupings based on time periods;
combine and organize said specific groupings into specific actions of said user over specific time periods;
compare prior and current data readings for said user using trained AI models to determine any changes in said user's condition.

2. The system according to claim 1, wherein said server is further configured to compare prior and current data readings for said user using trained diagnosis AI models to determine one or more conditions of said user.

3. The system according to claim 1, wherein said server is further configured to create a unique hash value based on at least one data reading, said hash value being for use as a user identifier.

4. The system according to claim 1, wherein said server is further configured to in conjunction with data relating to said user's treatment regimen, compare prior and current data readings for said user using trained treatment AI models to determine effects of said treatment regimen on one or more conditions of said user.

5. The system according to claim 2, wherein said server is configured to pass differences between said prior and current data readings through different trained diagnosis AI models, each diagnosis AI model being for determining if said differences indicate a presence of one or more conditions in said user.

6. The system according to claim 4, wherein said server is configured to pass differences between said prior and current data readings through different trained treatment AI models to determine gait-based side effects of said user's treatment regimen.

7. The system according to claim 1, wherein said server is configured to:
generate reports for said user's conditions based on analyses of said data readings;
transmit said reports to health professionals.

8. The system according to claim 3, wherein said unique hash value is based on one or more data readings from an immediately previous session with said user, said unique hash value being compared with a unique hash value derived from one or more current data readings.

9. The system according to claim 1, wherein trained AI models to be used in assessing said user's conditions are predetermined and based on at least one of:
said user's previous diagnosis;
said user's previously prescribed medications;
said user's previously assessed condition; and
said user's previously prescribed treatment regimen.

10. The system according to claim 1, wherein said data readings are used by said server to determine one or more of:
stride length;
cadence;
gait speed;
stride variability;
center of force;
a balance score;
load-bearing weight;
standing weight;
step count; and
weight biomarker shifts.

11. The system according to claim 1, wherein said server is configured to monitor outputs of one or more of said trained AI models for automated model drift detection, wherein, when statistically significant deviations from predefined limits are detected in said outputs, said server triggers an alert.

12. The system according to claim 1, wherein said server is configured to:
ingest data readings and data analyses outputs for said user; and
calculate dynamic risk scores based on said data readings and said data analyses outputs for use in continuous underwriting of health or life insurance coverage for said user.

13. The system according to claim 1, the wearable device comprises an insole for use in a user's shoe.

14. The system according to claim 1, further comprising a device abstraction layer, the device abstraction layer being configured to normalize telemetry from heterogeneous devices into a common schema for an analytics pipeline implemented by said system.

15. A method for assessing a user's condition, the method comprising:
a) receiving data from a wearable device, said data comprising current data readings for said user;
b) retrieving said user's stored data based on said data received from said wearable device, said stored data comprising prior data readings for said user;
c) processing said data to compare prior and current data readings for said user to determine any changes in said user's condition;
wherein step c) is executed using trained AI models.

16. The method according to claim 15, further comprising determining an identity hash based on said data received in step a) to determine said user's identity.

17. The method according to claim 16, wherein said identity hash is used to retrieve said user's stored data.

18. The method according to claim 15, further comprising determining said user's treatment regimes from said user's stored data.

19. The method according to claim 15, further comprising determining said user's diagnosed conditions based on said user's stored data.

20. The method according to claim 18, wherein said trained AI models determine an effectiveness of said user's treatment regimes on said user's condition based on said processing of said data.

21. The method according to claim 19, wherein said trained AI models determine a progression or a regression of said user's condition based on said processing of said data.
